(19) United States
(12) Patent Application Publication
(10) Pub. No.: US 2007/0124872 A1
(43) Pub. Date: Jun. 7, 2007

(54) Method of coloring with capped diazotized compound and coupling component

(76) Inventors: Victor Paul Eliu, Lorrach (DE); Beate Frohling, Steinen (DE); Dominique Kauffmann, Illzach (FR)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/582,750

(22) PCT Filed: Dec. 8, 2004
(86) PCT No.: PCT/EP04/53335

§ 371 Date: Jun. 14, 2006

(30) Foreign Application Priority Data
Dec. 19, 2003 (EP) 03104814.3

(51) Int. Cl. A61K 8/49

(52) U.S. Cl. 8/406

(57) ABSTRACT

The present invention relates to a method of coloring porous material, which comprises contacting the material being colored, with a) a capped diazonium compound of formula (1) wherein A⁺ is a cationic radical of an organic compound, B is a radical of an unsubstituted or substituted, aliphatic or aromatic amine, An is an anion, and b) optionally a coupling component. Further, the present invention relates to novel compounds and compositions thereof.

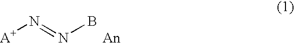

The present invention relates to a method of coloring porous material, for example metal, wood or keratin-containing fibres, especially human hair, using capped diazonium compounds and a coupling component.

U.S. patent application Ser. No. 10/469,619 describes a method of coloring hair with capped diazonium compounds and coupling components.

There is a need for coloring hair by such methods that damaging and undesired adverse effect can be minimized.

The problem underlying the present invention was to provide a method for coloring porous material, especially keratin-containing fibres, which allows to decrease the undesired adverse effects.

The present invention relates to a method of coloring porous material, which comprises contacting the material being colored, with

a) a capped diazonium compound of formula

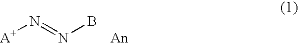

wherein

A⁺ is a cationic radical of an organic compound,

B is a radical of an unsubstituted or substituted, aliphatic or aromatic amine,

An is an anion,

and

b) optionally a coupling component.

Preferrably, A⁺ is a cationic radical of unsubstituted phenyl; naphthyl; thiophenyl; 1,3-thiazolyl; 1,2-thiazolyl; 1,3-benzothiazolyl; 2,3-benzothiazolyl; imidazolyl; 1,3,4-thiadiazolyl; 1,3,5-thiadiazolyl; 1,3,4-triazolyl; pyrazolyl; benzimidazolyl; benzopyrazolyl; pyridinyl; quinolinyl; pyrimidinyl; isoxazolyl; aminodiphenyl; aminodiphenylether and azobenzenyl or A+ is cationic radical of a phenyl, naphthyl, thiophenyl, 1,3-thiazolyl, 1,2-thiazolyl, 1,3-benzothiazolyl, 2,3-benzothiazolyl, imidazolyl, 1,3,4-thiadiazolyl, 1,3,5-thiadiazolyl, 1,3,4-triazolyl, pyrazolyl, benzimidazolyl, benzopyrazolyl, pyridinyl, quinolinyl, pyrimidinyl and isoxazolyl, aminodiphenyl, aminodiphenylether and azobenzenyl, each of which is mono- or poly-substituted by C₁-C₄alkyl, C₁-C₄alkoxy, C₁-C₄alkylthio, quaternised ammonium radicals, halogen, e.g. fluorine, bromine or chlorine, nitro, trifluoromethyl, CN, SCN, C₁-C₄alkylsulfonyl, phenylsulfonyl, benzylsulfonyl, di-C₁-C₄alkylaminosulfonyl, C₁-C₄alkyl-carbonylamino, C₁-C₄alkoxysulfonyl or by di-(hydroxy-C₁-C₄alkyl)-aminosulfonyl, or A⁺ is a cationic radical residue of an organic dye.

Suitable anions, An, are both inorganic and organic anions, for example halide, such as chloride, bromide or iodide, hydroxid, sulfate, hydrogen sulfate, C₁-C₆alkyl sulfonate, such as methyl sulfonate or ethy sulfonate, C₁-C₆alkyl sulfate, C₁-C₆alkyl substituted or unsubstituted arylsulfonate, such as 4-toluylsulfonate, formate, such as methyl sulfate or ethy sulfate, acetate, tartrate, oxalate, and lactate.

Preferred anions are chloride, hydrogen sulfate, sulfate, methosulfate or acetate.

Preferred is the method according the present invention, which comprises contacting the material being colored, with

a) a capped diazonium compound of formula (1)

wherein

A⁺ is a cationic radical of unsubstituted phenyl; naphthyl; thiophenyl; 1,3-thiazolyl; 1,2-thiazolyl; 1,3-benzothiazolyl; 2,3-benzothiazolyl; imidazolyl; 1,3,4-thiadiazolyl; 1,3,5-thiadiazolyl; 1,3,4-triazolyl; pyrazolyl; benzimidazolyl; benzopyrazolyl; pyridinyl; quinolinyl; pyrimidinyl; isoxazolyl; aminodiphenyl; aminodiphenylether and azobenzenyl, or A+ is cationic radical of a phenyl, naphthyl, thiophenyl, 1,3-thiazolyl, 1,2-thiazolyl, 1,3-benzothiazolyl, 2,3-benzothiazolyl, imidazolyl, 1,3,4-thiadiazolyl, 1,3,5-thiadiazolyl, 1,3,4-triazolyl, pyrazolyl, benzimidazolyl, benzopyrazolyl, pyridinyl, quinolinyl, pyrimidinyl and isoxazolyl, aminodiphenyl, aminodiphenylether and azobenzenyl, each of which is mono- or poly-substituted by C₁-C₄alkyl, C₁-C₄alkoxy, C₁-C₄alkylthio, quaternised ammonium radicals, halogen, e.g. fluorine, bromine or chlorine, nitro, trifluoromethyl, CN, SCN, C₁-C₄alkylsulfonyl, phenylsulfonyl, benzylsulfonyl, di-C₁-C₄alkylaminosulfonyl, C₁-C₄alkyl-carbonylamino, C₁-C₄alkoxysulfonyl or by di-(hydroxy-C₁-C₄alkyl)-aminosulfonyl, or

A⁺ is a cationic radical residue of an organic dye, and

B is a radical of formula —NR₆₅R₆₆, wherein R₆₅ is hydogen; or unsubstituted linear or branched

C₁-C₆alkyl; or linear or branched C₁-C₆alkyl, which is substituted by one or more identical or different substituent selected from the group consisting of C₁-C₄alkoxy, COOH, COO⁻, COOC₁-C₂alkyl, SO₃H, SO₃⁻, NH₂, CN, halogen and OH, O⁻; and R₆₆ is unsubstituted linear or branched C₁-C₆alkyl; or linear or branched C₁-C₆alkyl, which is substituted by one or more identical or different substituent selected from the group consisting of C₁-C₄alkoxy, COOH, COO⁻, COOC₁-C₂alkyl, SO₃H, SO₃⁻, NH₂, CN, halogen and OH, O⁻; or

B is a radical of unsubstituted aniline; or a radical of unsubstituted aminonaphthalene; the radical of aniline or aminonaphthalene, wherein the phenyl or the naphthyl ring is substituted by one or more identical or different substituent selected from the group consisting of COOH, COO⁻, SO₃H, SO₃⁻, CN, halogen, SO₂C₁-C₂alkyl; or unsubstituted linear or branched C₁-C₄alkyl, linear or branched C₁-C₄alkyl, substituted by OH, O—, carboxy, COO—, COC₁-C₂alkyl or SO₂—N(C₁-C₄alkyl)-(CH₂)₁₋₄SO₃H and wherein the amino radical is substituted by hydrogen, unsubstituted linear or branched C₁-C₄alkyl; or linear or branched C₁-C₄alkyl, substituted by OH, O⁻, COOH or COO⁻;

An is an anion,

and

b) a optionally coupling component.

More preferred is the method according to the present invention, wherein A⁺ is a cationic radical of unsubstituted phenyl; naphthyl; thiophenyl; 1,3-thiazolyl; 1,2-thiazolyl; 1,3-benzothiazolyl; 2,3-benzothiazolyl; imidazolyl; 1,3,4-thiadiazolyl; 1,3,5-thiadiazolyl; 1,3,4-triazolyl; pyrazolyl; benzimidazolyl; benzopyrazolyl; pyridinyl; quinolinyl; pyrimidinyl; isoxazolyl; aminodiphenyl; aminodiphenylether and azobenzenyl or

A⁺ is cationic radical of a phenyl, naphthyl, thiophenyl, 1,3-thiazolyl, 1,2-thiazolyl, 1,3-benzothiazolyl, 2,3-benzothiazolyl, imidazolyl, 1,3,4-thiadiazolyl, 1,3,5-thiadiazolyl, 1,3,4-triazolyl, pyrazolyl, benzimidazolyl, benzopyrazolyl, pyridinyl, quinolinyl, pyrimidinyl and isoxazolyl, aminodiphenyl, aminodiphenylether and azobenzenyl, each of which is mono- or poly-substituted by C₁-C₄alkyl, C₁-C₄alkoxy, C₁-C₄alkylthio, halogen, e.g. fluorine, bromine or chlorine, nitro, trifluoromethyl, CN, SCN, C₁-C₄alkylsulfonyl, phenylsulfonyl, benzylsulfonyl, di-C₁-C₄alkylaminosulfonyl, C₁-C₄alkyl-carbonylamino, C₁-C₄alkoxysulfonyl or by di-(hydroxy-C₁-C₄alkyl)-aminosulfonyl, or

A⁺ is a cationic radical of an organic dye selected from anthraquinon dye, acridine dye, azo dye, azomethin dye, hydrazomethin, benzodifuranone dye, coumarin dye, diketopyrrolopyrrol dye, dioxaxine dye, diphenylmethane dye, formazan dye, indigoid dye, indophenol, naphtalimide dye, naphthoquinone dye, nitroaryl dye, merocyanine dye, methin dye, oxazine dye, perinone dye, perylene dye, pyrenequinone dye, phthalocyanine dye, phenazine dye, quinoneimine dye, quinacridone dye, quinophthalone dye, styryl dye, triphenylmethan dye, xanthene dye, thiazine dye and thioxanthene dye, and especially preferred cationic radical of an organic dye, azo dye, azomethin dye, hydrazomethin dye, merocyanine dye, methin dye and styryl dye;

B is a radical of formula —NR₆₅R₆₆, wherein R₆₅ is hydrogen; or unsubstituted linear or branched C₁-C₆alkyl; or a linear or branched C₁-C₆alkyl, which is substituted by one or more identical or different substituent selected from the group consisting of OC₁-C₄alkyl, COOH, COO⁻, COOC₁-C₂alkyl, SO₃H, SO₃⁻, NH₂, CN, halogen and OH, O⁻; and R₆₆ is unsubstituted linear or branched C₁-C₆alkyl; or linear or branched C₁-C₆alkyl, which is substituted by one or more identical or different substituent selected from the group consisting of OC₁-C₄alkyl, COOH, COO⁻, COOC₁-C₂alkyl, SO₃H, SO₃⁻, NH₂, CN, halogen and OH, O⁻. Preferably, B is —NR₆₅R₆₆, wherein R₆₅ is unsubstituted linear or branched C₁-C₆alkyl, linear or branched C₁-C₆alkyl, which is substituted by one or more identical or different substituent selected from the group consisting of COOH, COO⁻. Most preferably B is —NR₆₅R₆₆, wherein R₆₅ is C₁-C₆alkyl, especially, methyl or ethyl, and R₆₆ is C₁-C₆alkyl, especially, methyl or ethyl, or methylCOO—, methylCOOH, ethylCOO⁻, ethylCOOH, propylCOO⁻.

A further preferred method of the present invention comprises contacting

a) a capped diazonium compound of formula

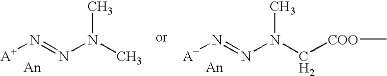

wherein

A⁺ is a cationic radical of an organic compound,

An is an anion,

and

b) optionally a coupling component.

Especially more preferred is the method according to any of the precedings claims, wherein A⁺ is a cationic radical of an organic dye selected from azo dye, azomethin dye, hydrazomethin dye, merocyanine dye, methin dye and styryl dye.

Suitable coupling components are, for example, the usual coupling components customarily used for azo dyes and known from the pertinent literature, e.g. coupling components from the benzene series, naphthalene series, open-chain methylene-active compounds, such as acylacetarylamides, and those of the heterocyclic series.

Such coupling components may carry further substituents, for example amino, alkylamino, dialkylamino, halogen, alkyl, alkoxy, aryl, especially phenyl or naphthyl, or aryloxy, but especially a group imparting water solubility, e.g. hydroxy, carboxy, sulfo or a quaternised ammonium radical.

The coupling components preferably carry one or two such groups imparting water solubility. Examples of suitable coupling components are as follows:

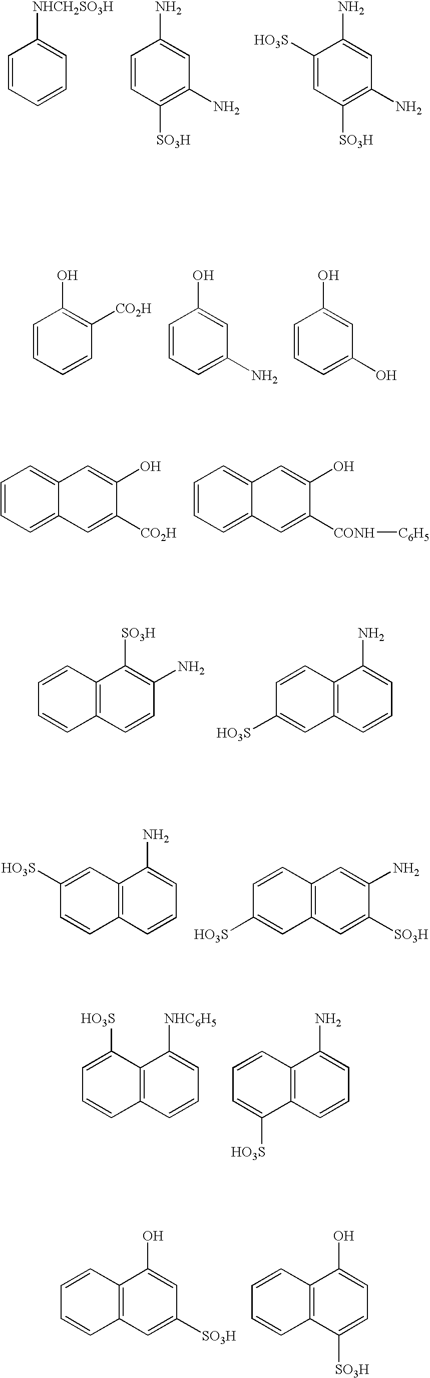

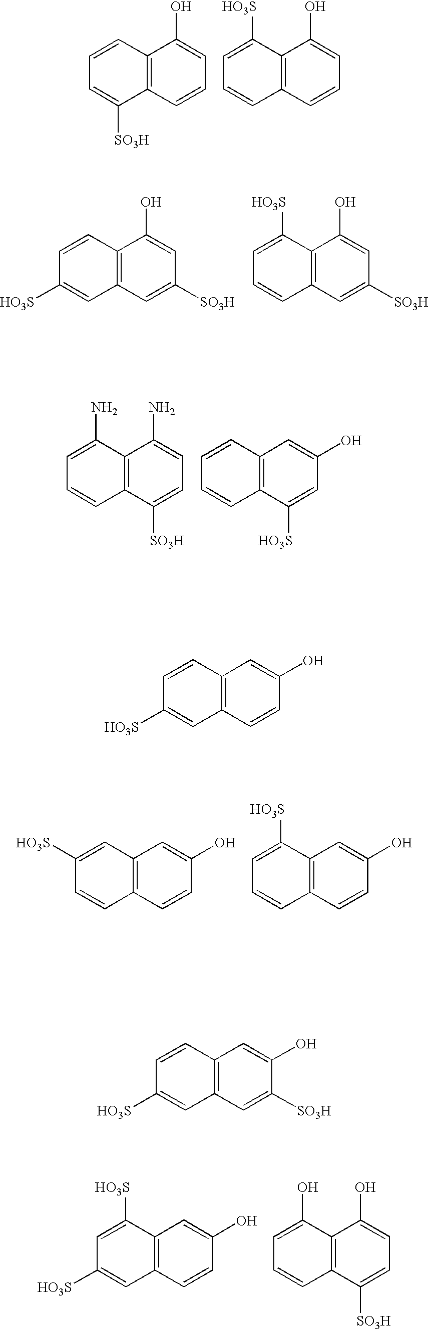

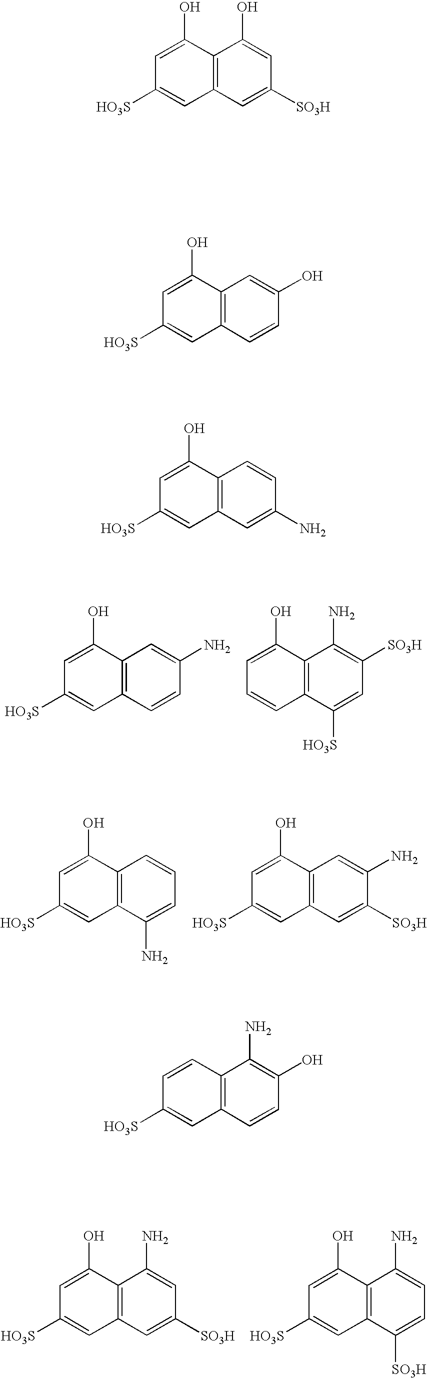

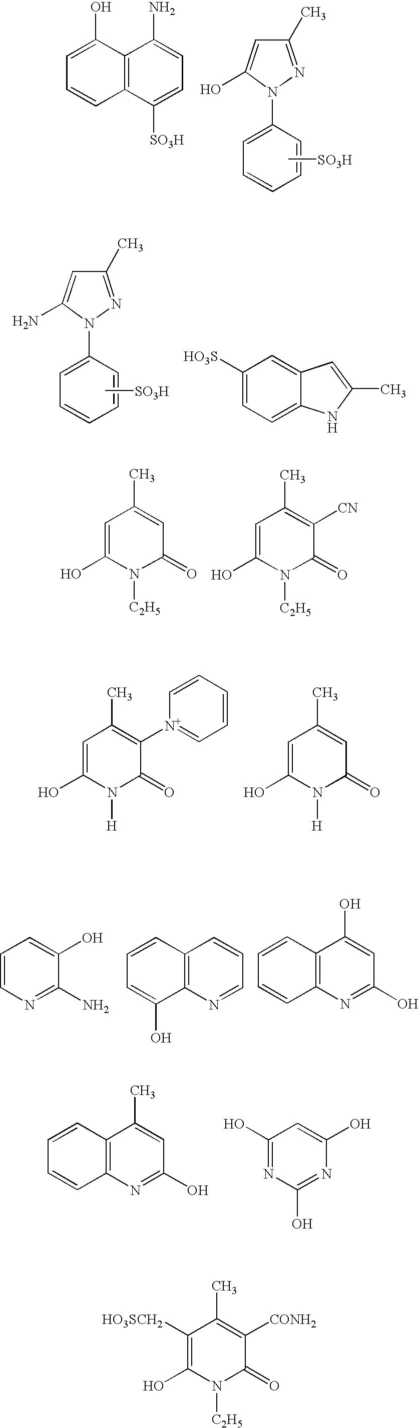

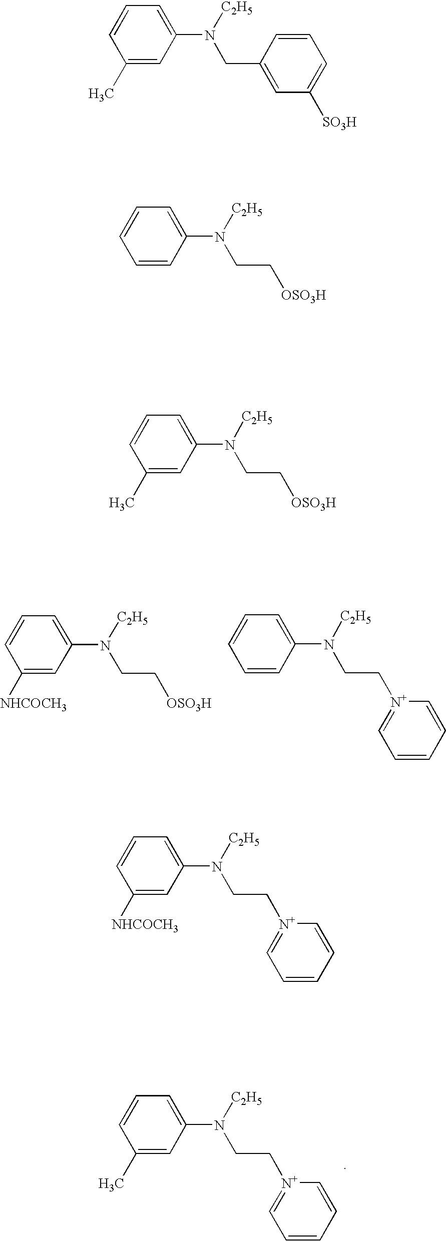

Preferred coupling component is an unsubstituted or substituted acylacetarylamide, phenol, naphthol, pyridine, quinolone, pyrazole, indole, diphenylamine, aniline, aminopyridine, pyrimidone, naphthylamine, aminothiazole, thiophene or hydroxypyridine.

Preferred substituents of coupling component are at least one amino, alkylamino, dialkylamino, halogen, alkyl, alkoxy, phenyl, naphthyl or aryloxy.

Further, preferred is the method of the present invention, wherein A⁺ is a cationic radical of a dye of formulae (7) and (8)

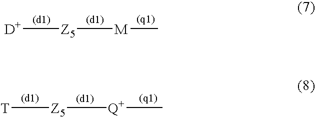

wherein

Z₅ is a biradical selected from:

—N═N—, —CR₆═N—, —N═CR₇—, —NR₈—N═CR₉—, —R₁₀C═N—NR₁₁—, —CR₆═CR₆—,

wherein

R₆, R₇, R₈, R₉, R₁₀ and R₁₁ are each independently of the other hydrogen, or unsubstituted or substituted C₁-C₁₄alkyl, allyl, —C₅-C₁₀aryl, -C₁-C₁alkylen(C₅-C₁₀aryl),

—C₅-C₁₀arylen-(C₁-C₁₀alkyl), and

D⁺ is a radical of a cationic, aromatic, substituted or unsubstituted heterocydic compound,

M is a biradical of an aromatic substituted or unsubstituted compound,

T is a radical of an aromatic substituted or unsubstituted compound, and

Q⁺ is a biradical of an aromatic, substituted or unsubstituted heterocyclic compound.

Preferrably, D⁺ is a radical of a cationic aromatic substituted or unsubstituted heterocyclic compound of formulae (9), (10), (10′), (10″), (11), (12) or (13)

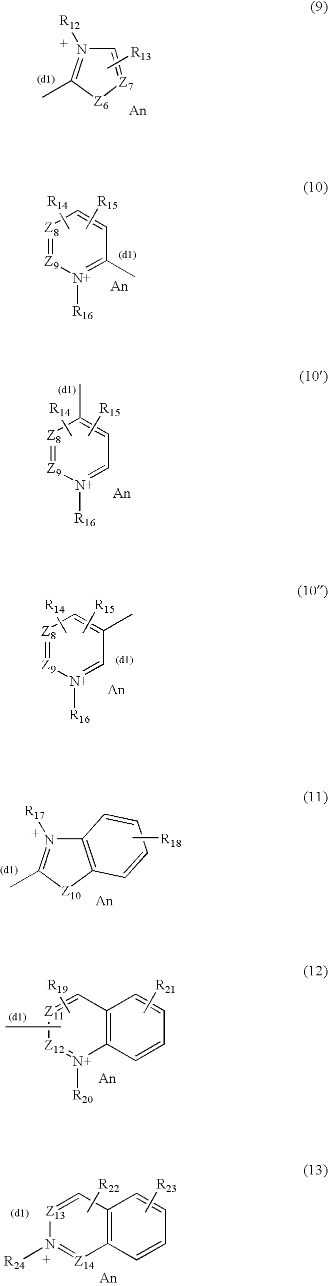

wherein

(d1) is a bond of formula (7);

and

Q⁺ is a biradical of a cationic aromatic substituted or unsubstituted heterocyclic compound of formulae (14), (14′), (15), (15′), (15″), (16), (17) or (18)

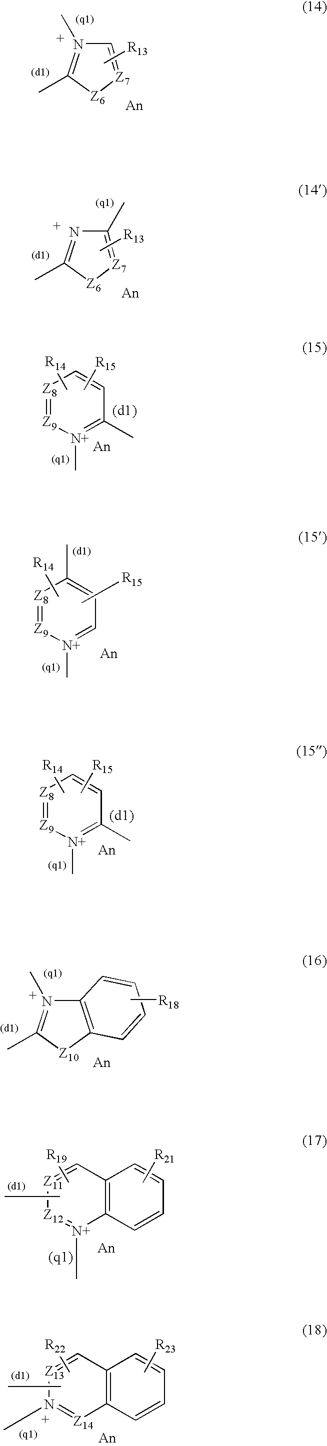

wherein

(d1) and (q1) are a bond to Z₅ of formula (8),

and

M is a biradical of formulae (19) or (20),

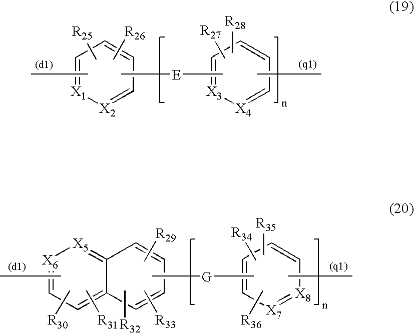

wherein

(d1) and (q1) are a bond of formula (7), and

T is a radical of compounds of formulae (21) or (22),

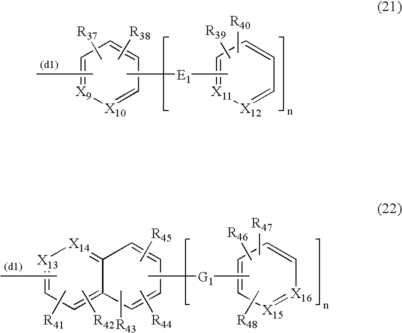

wherein

(d1) is a bond of formula (8), and

wherein

X_{1,} X_{2,} X_{3,} X_{4,} X_{5,} X_{6,} X_{7,} X_{8,} X_{9,} X_{10,} X_{11,} X_{12,} X_{13,} X_{14,} X₁₅ and X₁₆ are independently from each other N or a radical of CR₄₉,

Z₆ is O or S or a radical of NR₅₀,

Z₇, Z₈, Z₉, Z₁₀, Z₁₁, Z₁₂, Z₁₃ and Z₁₄ are independently from each other N or a radical of CR₅₁;

E, E₁, G and G₁ are independently from each other —O—, —S—, —(SO₂)—, —C₁-C₁₀alkylen or —(NR₅₂)—;

R_{13,} R_{14,} R_{15,} R_{18,} R₁₉, R₂₁, R₂₂, R₂₃, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉ and R₅₁ are independently from each other hydrogen, halogen, C₁-C₁₄alkyl, which is saturated or unsaturated, linear or branched, substituted or unsubstituted, or interrupted or uninterrupted with heteroatoms; a radical of phenyl, which substituted or unsubstituted; a radical of carboxylic acid; a radical of hydroxy, nitril, C₁-C₁₆alkoxy, (poly)-hydroxy-C₂-C₄-alkoxy, carboxylic acid, sulfonic acid; halogen, sulfonylamino, SR₆₀, NHR₆₃ or NR₅₄R₅₅, OR₆₁, SO₂, COOR₆₂, NR₅₆COR₅₈, CONR₅₇; and

R_{12,} R_{16,} R_{17,} R_{20,} R₂₄, R₅₀, R₅₂, R₅₃, R₅₄, R₅₅, R₅₆, R₅₇, R₅₈, R₆₀, R₆₁ and R₆₂ are each independently of the other hydrogen, unsubstituted or substituted C₁-C₁₄alkyl, allyl, —C₅-C₁₀arylen-(C₁-C₁₀alkyl), —C₁-C₁₀alkylen(C₅-C₁₀aryl), C₅-C₁₀aryl, and

An is an anion.

More preferred is D⁺ a radical of a cationic aromatic substituted or unsubstituted heterocyclic compound of formulae (23), (24), (24a), (25), (26), (26a) or (27)

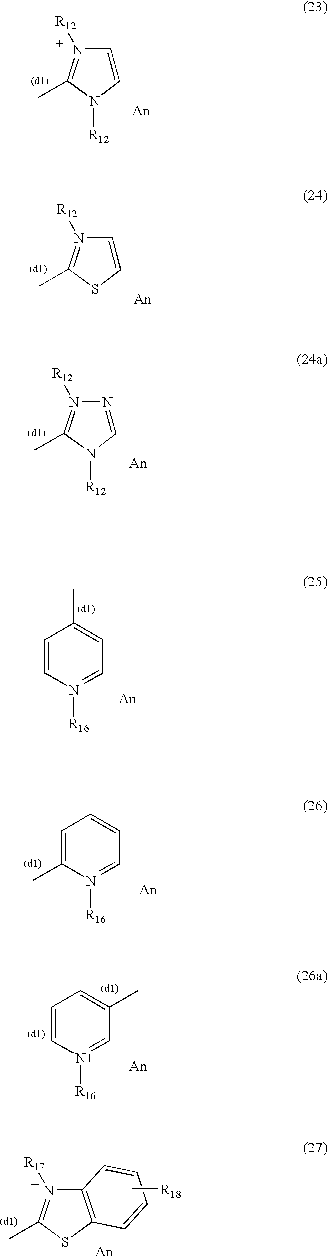

wherein

(d1) and (q1) are a bond of formula (7), and

An, R₁₂, R₁₆, R₁₇ and R₁₈ have the same meaning as given above

and

Q⁺ is a biradical of a cationic aromatic substituted or unsubstituted heterocyclic compound of formulae (28), (28a), (29), (29a), (30), (31), (31 a) or (32)

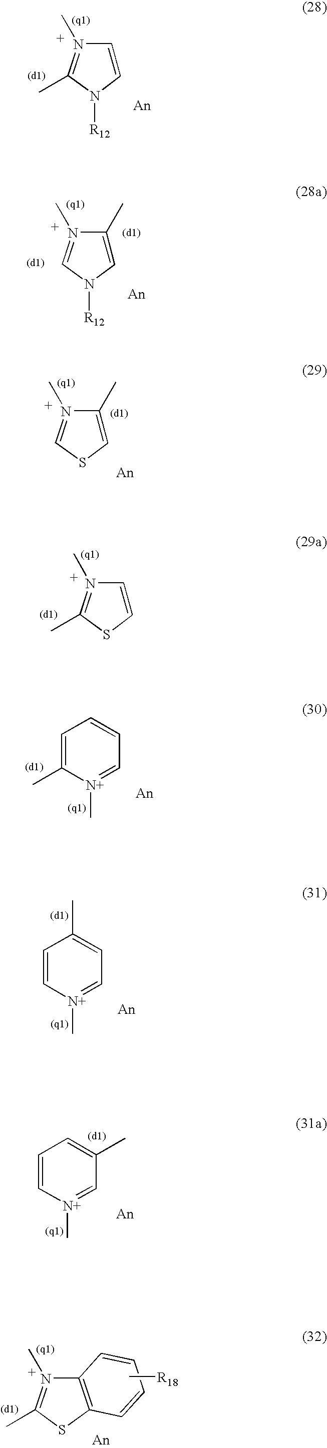

wherein

(d1) and (q1) are bond of formula (8), and

An, R₁₂ and R₁₈ have the same meaning as given above

and

M is a biradical of formulae (33), (33a) or (33b),

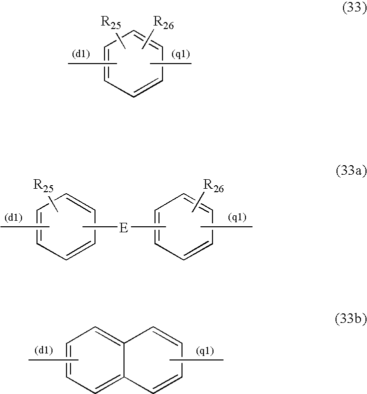

wherein

(d1) and (q1) are bond of formula (7), and

E, R₂₅ and R₂₆ have the same meaning as given above;

and

T is a radical of formulae (34) or (34a),

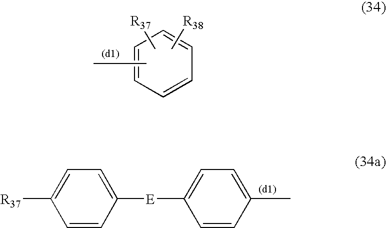

wherein

R₃₇, R₃₈ and E has the same defintion and preferences as given above, and

(d1) is a bond of compound of formula (8).

More preferred is a method according to the present invention, which comprises contacting the material being colored, with

a) at least a single cationic capped diazonium compound selected from the group of compounds of the following formulae

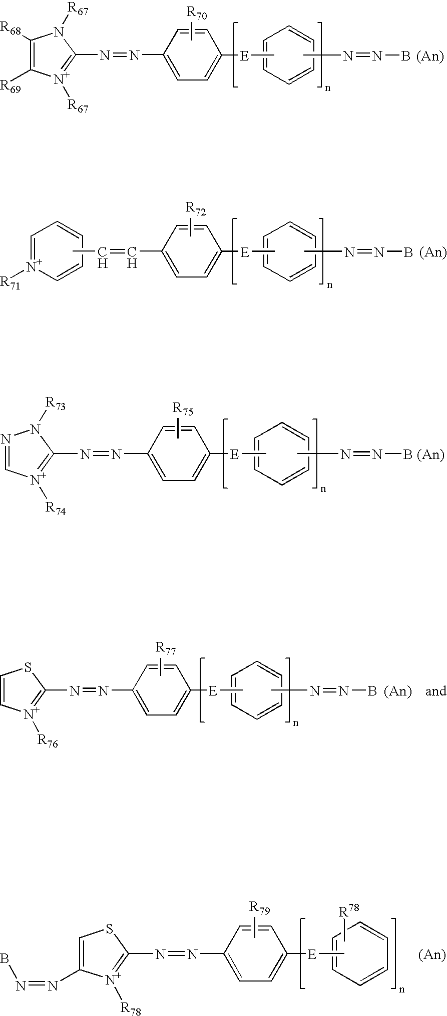

wherein

E is —O—, —S—, —(SO₂)—, CR₈₀ or a radical of —NR₈₁)—;

R₇₀, R₇₂, R₇₅, R₇₇, R₇₈, R₇₉, R₈₀ and R₈₁ are independently from each other hydrogen,

C₁-C₁₆alkyl, which is saturated or unsaturated, linear or branched, substituted or unsubstituted, or interrupted or uninterrupted with heteroatoms, such as, by hydroxy, nitril, amino, C₁-C₂ alkoxy, (poly)-hydroxy-C₂-C₄-alkoxy, di-C₁-C₂ alkylamino, carboxylic acid, sulfonic acid; a radical of phenyl, which substituted or unsubstituted; a radical of carboxylic acid; a radical of sulfonylamino, S, NH or N(C₁-C₄alkyl), O, halogen, SO₂, COO, OCO, NHCO, CONH, CON(C₁-C₄alkyl) or N(C₁-C₄alkyl)CO; or are independently from each other an aliphatic or aromatic, substituted;

R₆₈ with R₆₉ have the same meaning as R₇₀, R₇₂, R₇₅, R₇₇, R₇₈, R₇₉, R₈₀ and R₈₁ as given above, or

R₆₈ with R₆₉ can build up an aromatic carbon cycle;

R₆₇, R₇₁, R₇₃, R₇₄, R₇₆ and R₇₈ are unsubstituted or substituted C₁-C₁₄alkyl, allyl, —C₅-C₁₀arylen-(C₁-C₁₀alkyl), —C₁-C₁₀alkylen(C₅-C₁₀aryl), C₅-C₁₀aryl;

B, An and n have the same meaning as given above,

and

b) a coupling component.

According to the invention alkyl, such as C₁-C₁₆alkyl, C₁-C₁₄alkyl, C₁-C₄alkyl or C₁-C₁₀alkylen can be substituted, unsubstituted, linear or branced, interrupted, not interrupted by at least one heteroatom such as —O—, —S—, —(SO₂)— or —(NR₆)—; from C5-upwards cyclic or noncyclic.

C₁-C₁₆alkyl is, for example, methyl, ethyl, propyl, isopropyl, n-butyl, sec-butyl, tert-butyl, n-pentyl, 2-pentyl, 3-pentyl, 2,2′-dimethylpropyl, cyclopentyl, cyclohexyl, n-hexyl, n-octyl, 1,1′,3,3′-tetramethylbutyl or 2-ethylhexyl, nonyl, decyl, undecy, dodecyl, tredecyl, tetradecyl, pentadecyl or hexadedy.

C₁-C₁₄alkyl is, for example, methyl, ethyl, propyl, isopropyl, n-butyl, sec-butyl, tert-butyl, n-pentyl, 2-pentyl, 3-pentyl, 2,2′-dimethylpropyl, cyclopentyl, cyclohexyl, n-hexyl, n-octyl, 1,1′,3,3′-tetramethylbutyl or 2-ethylhexyl, nonyl, decyl, undecy, dodecyl, tredecyl, tetradecyl.

C₁-C₄alkyl is, for example, methyl, ethyl, propyl, isopropyl, n-butyl, sec-butyl, tert-butyl.

C₁-C₁₀alkylen is, for example, methylen, ethylen, propylen, isopropylen, n-butylen, sec-butylen, tert-butylen, n-pentylen, 2-pentylen, 3-pentylen, 2,2′-dimethylpropylen, cyclopentylen, cydohexylen, n-hexylen, n-octylen, 1,1′,3,3′-tetramethylbutylen or 2-ethylhexylen, nonylenor decylen.

COOC₁-C₂alkyl is, for example, COOmethyl, COOethyl.

C₁-C₁₆alkoxy is, for example, methoxy, ethoxy, propoxy, isopropoxy, n-butoxy, sec-butoxy, tert-butoxy, n-pentoxy, 2-pentoxy, 3-pentoxy, 2,2′-dimethylpropoxy, cyclopentoxy, cyclohexoxy, n-hexoxy or n-octoxy;

C₁-C₄alkoxy is, for example, methoxy, ethoxy, propoxy, isopropoxy, n-butoxy, sec-butoxy or tert-butoxy;

(poly)-hydroxy-C₂C₄-alkoxy is, for example, (poly)-hydroxy-ethoxy(poly)-hydroxy-propoxy, (poly)-hydroxy-butoxy;

C₁-C₄alkylthio is, for example, methylthio, ethylthio, propylthio, isopropylthio, n-butylthio, sec-butylthio or tert-butylthio;

quaternised ammonium radicals are substituted by four substitutents selected from the group of substitutents, which are hydrogen, or C₁-C₁₆alkyl,

C₅-C₁₀aryl, allyl;

halogen is, for example, fluor, chlor, brom or jod;

C₁-C₄alkylsulfonyl is, for example, methylsulfonyl, ethylsulfonyl, propylsulfonyl, isopropylsulfonyl, n-butylsulfonyl, sec-butylsulfonyl or tert-butylsulfonyl.

di-C₁-C₄alkylaminosulfonyl is, for example, di-methylaminosulfonyl, di-ethylaminosulfonyl, di-propylaminosulfonyl, di-isopropylaminosulfonyl, di-n-butylaminosulfonyl, di-sec-butylaminosulfonyl, di-tert-butylaminosulfonyl.

C₁-C₄alkyl-carbonylamino is, for example, methyl-carbonylamino, ethyl-carbonylamino, propyl-carbonylamino, isopropyl-carbonylamino, n-butyl-carbonylamino, sec-butyl-carbonylamino, tert-butyl-carbonylamino.

C₁-C₄alkoxysulfonyl is, for example, methoxy sulfonyl, ethoxy sulfonyl, propoxy sulfonyl, isopropoxy sulfonyl, n-butoxy sulfonyl, sec-butoxy sulfonyl, tert-butoxy sulfonyl di-(hydroxy-C₁-C₄alkyl)-aminosulfonyl,

SO₂—N(C₁-C₄alkyl)-(CH₂)₁₋₄SO₃H

—C₅-C₁₀aryl is for example substituted or unsubstituetd cyclodienylanion, phenyl or naphthyl, and preferably aryl is phenyl.

Aromatic carbon cycle is for example-C₅-C₁₀aryl.

Aralkyl is for example (C₁-C₄alkyl)phenyl, methylphenyl, ethylphenyl, propylphenyl, isopropylphenyl, n-butylphenyl, sec-butylphenyl, tert-butylphenyl, preferably benzyl.

Especially preferred in the present invention is the method of coloring porous material, by using at least a single capped diazonium compound selected from the group of compounds of the following formulae

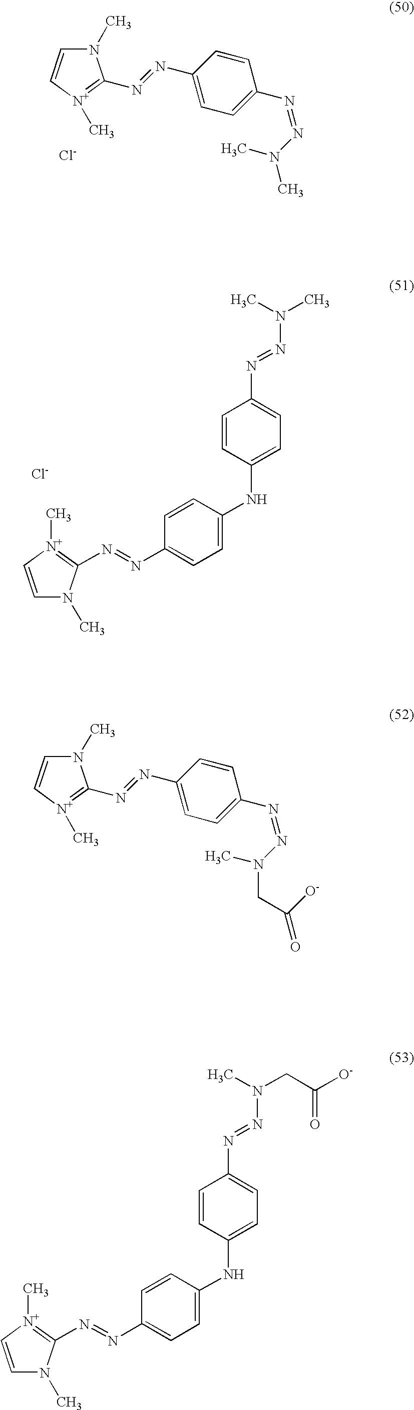

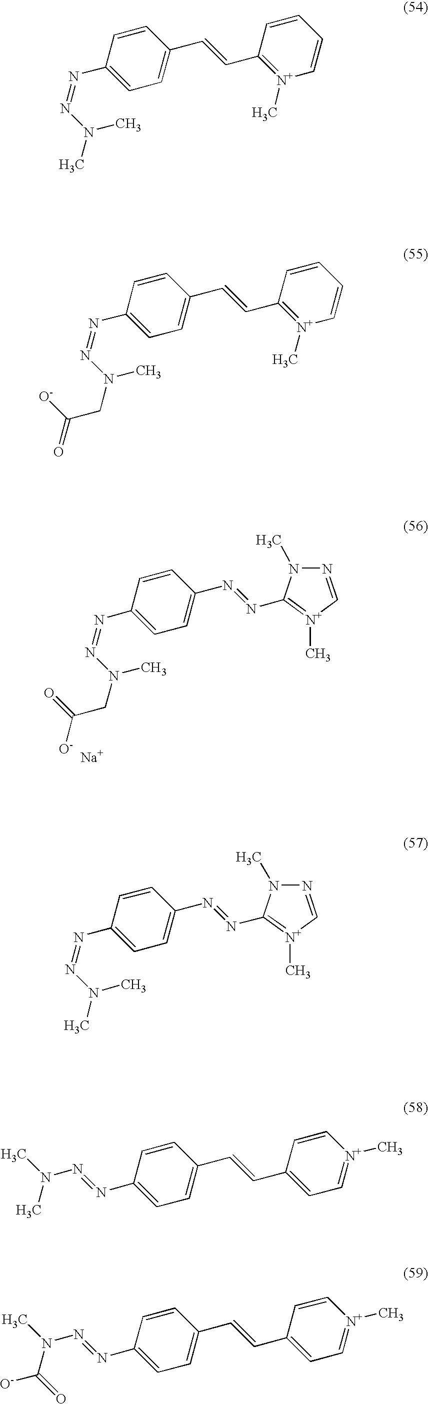

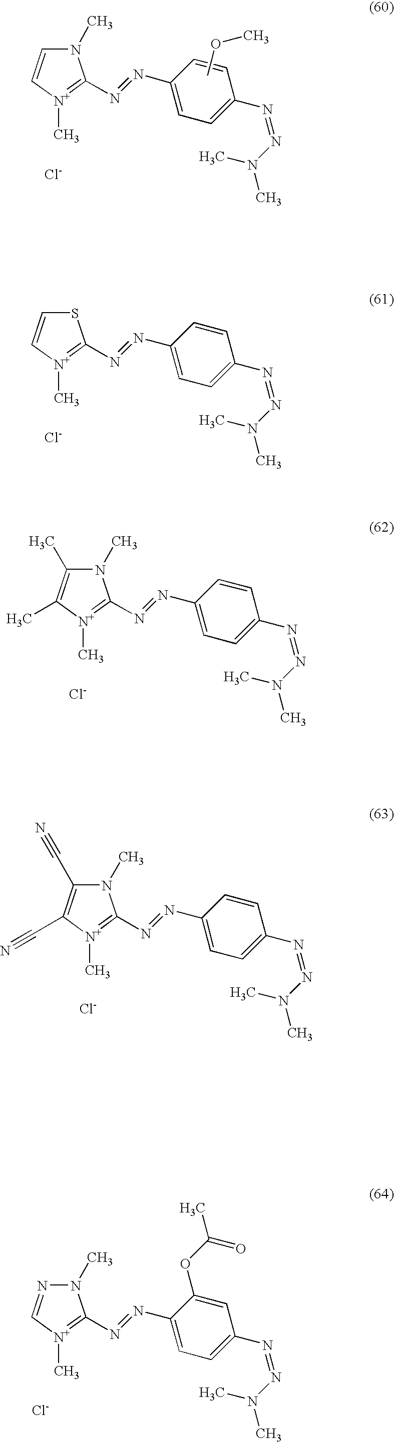

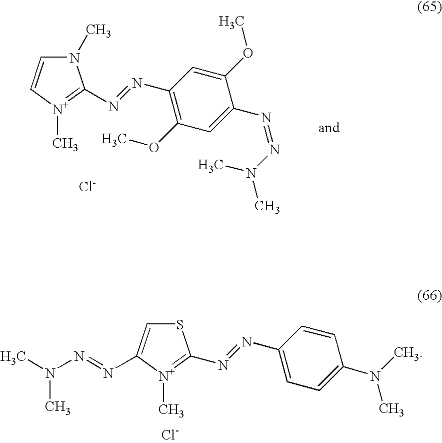

The first stage of the coloring method according to the invention comprises applying to the material being colored, in any desired order successively, or simultaneously, a capped diazonium compound and a water-soluble coupling component under conditions such that, initially, coupling does not take place. That is accomplished, for example, by immersing the material in a solution comprising the capped diazonium compound or the coupling component and then, optionally after rinsing and intermediate drying, immersing the material in a solution of the second component. Preferably, however, the capped diazonium compound and the coupling component are contained together in one solution. It is also possible for the solutions in question to be applied to the material by means of spraying or similar means, although it must be ensured that there is adequate penetration unless it is desired to colour only the upper layers. During that first stage the diazonium compound and the coupling component should not yet react with one another, which is achieved preferably by maintaining a pH of from 8 to 12, especially from 9 to 11.

In the second stage, the diazonium compound and the coupling component are then caused to react, preferably by lowering the pH to a value of from 5 to 2, especially from 3 to 4. Lowering the pH is achieved in conventional manner by adding an acid, such as tartaric acid or citric acid, a citric acid gel, a suitable buffer solution or by means of an acid dye.

The ratio of the amount of alkaline coloring composition applied in the first stage to that of acid coloring composition applied in the second stage is preferably about from 1:3 to 3:1, especially about 1:1.

The contact time is preferably about from five to thirty minutes in each case, especially from 10 to 20 minutes in each case.

The colored material is then finished in customary manner, for example by rinsing with water and then drying.

The method according to the invention is suitable for all-over coloring of the hair that is to say when coloring the hair on a first occasion and also for re-coloring subsequently.

A preferred embodiment of the method according to the invention relates to the coloring of porous material by bringing the material being colored into contact with a capped diazonium compound and a water-soluble coupling component, in any desired order successively, or simultaneously,
a) under alkaline conditions and optionally in the presence of a further dye, preferably an oxidation dye, or a cationic, anionic or uncharged direct dye, especially a cationic dye selected from the group of the cationic dyes as described in WO 95/01772 and WO 01/66646, and
then subjecting the material being colored to treatment with acid, or
b) under alkaline conditions, and
then subjecting the material being colored to treatment with acid, optionally in the presence of a further dye, preferably an oxidation dye, or a cationic, anionic or uncharged direct dye, especially a cationic dye selected from the group of the cationic dyes as described in WO 95/01772 and WO 01/66646, or
c) under alkaline conditions in the presence of an oxidising agent, and optionally in the presence of a further dye, preferably an oxidation dye, or a cationic, anionic or uncharged direct dye, especially a cationic dye selected from the group of the cationic dyes as described in WO 95/01772 and WO 01/66646 and then subjecting the material being colored to treatment with acid.

A further embodiment of the present invention is compounds of formula (1)

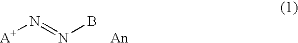

wherein

A⁺ is a cationic radical of an organic compound,

B is a radical of an unsubstituted or substituted, aliphatic or aromatic amine,

An is an anion, with the proviso that A⁺ is not a radical of formula

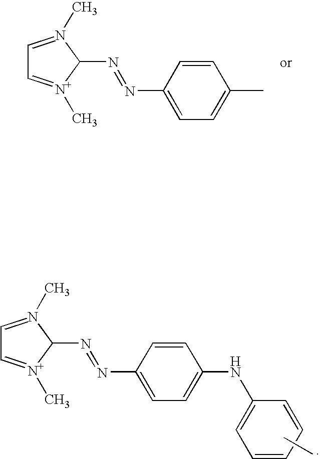

In addition, the present invention concerns compositions comprising at least a single capped diazonium compound of formula (1) as defined above and a coupling component.

Preference is given to compositions comprising in addition a direct dye, and/or at least a single oxidative dye and/or an oxidative agent.

More preference is given to compositions in form of a shampoo, conditioner, gel or emulsion.

One preferred embodiment of the present invention concerns the method according to the invention for dyeing or tinting human hair.

The compositions of the present invention comprising compound (1) have the same preferences for compound (1) as described in the method according to the present invention above.

In the methods according to the invention, whether or not coloring is to be carried out in the presence of a further dye will depend upon the colour shade to be obtained.

In the context of the present invention, the expression “alkaline conditions” denotes a pH in the range from 8 to 10, preferably 9-10, especially 9.5-10.

Further, acids used in coloring methods of porous material, especially hair, according to the present invention are for example tartaric acid or citric acid, a citric acid gel, a suitable buffer solution with optionally an acid dye.

Adding bases, for example sodium carbonate, ammonia or sodium hydroxide, to the hair or to the dye precursors, the capped diazonium compound and/or the water-soluble coupling component, or to coloring compositions comprising the dye precursors, customarily achieve the alkaline conditions.

In the context of the present invention, oxidizing agents are understood to be any oxidizing agent customarily used for oxidative hair coloring, for example dilute hydrogen peroxide solutions, hydrogen peroxide emulsions or hydrogen peroxide gels, alkaline earth metal peroxides, organic peroxides, such as urea peroxides, melamine peroxides, or alkalimetalbromat fixations are also applicable if a shading powder on the basis of semi-permanent, direct hair dyes is used.

An oxidizing agent, which can be added to the coloring compositions according to the invention comprise an oxidizing agent and optionally a base.

The oxidising agents are used in appropriate stoichiometric amounts that correspond to the total molar amounts of oxidation dye precursors. Preferred oxidizing agent is hydrogen peroxide, preferred in about 2 to 30% by weight, more preferred in 3 to 20% by weight, and most preferred in 6 to 12% by weight of the total weight of a watery composition such as a solution, dispersion, a gel or emulsion.

An oxidizing agents may be present in the coloring compositions according to the invention preferably in an amount of from 0.01% to 6%, especially from 0.01% to 1%, based on the total dyeing composition.

The method according to the invention is used for coloring porous material, for example wood, glass fibres, aluminium, cotton, paper, natural or synthetic polyamides, e.g. leather, wool, nylon or perlon, but especially keratin-containing fibres and more especially for coloring hair. The hair may be the hair of wigs or, especially, the living hair of animals and, more especially, humans.

The invention relates also to coloring compositions for carrying out the method according to the invention, which compositions comprise

a) a compound of formula (1) indicated hereinbefore,

b) a medium for adjusting the pH,

c) water
and, optionally,

d) further additives.

Preferred compositions comprise

a) a compound of formula (1) indicated hereinbefore,

b) a medium for adjusting the pH,

c) water,

d) a coupling component
and, optionally,

e) further additives.

Especially preferred compositions comprise
a) a compound of formula (1) indicated hereinbefore,
b) a medium for adjusting the pH,
c) water,
d) a coupling component,
e) a further dye, preferably an oxidation dye, or a cationic, anionic or uncharged direct dye, especially a cationic dye selected from the group of the cationic dyes as described in WO 95/01772 and WO 01/66646,
   and, optionally,
f) further additives.

Special preference is given to coloring compositions for the coloring of hair. Further additives that are suitable for such compositions include additives that are customary in hair-coloring, for example further dyes, surfactants, solvents, perfumes, polymeric adjuvants, thickeners and light stabilisers.

The dyes of formula (1) according to the invention are suitable for dyeing organic material, such as keratin, wool, leather, silk, paper, cellulose or polyamides, especially keratin-containing fibers, cotton or nylon, and preferably human hair.

The multiplicity of shades of the dye, which results by the method according to the present invention, can be increased by combination with other dyes.

The present invention relates also to the coloration of hair with a dye of formula (1) according to the present invention, and at least a single further dye.

One embodiment of the method of present invention concerns the use of a combination of the dye of formula (1) with dyes of the same or different class of dyes, especially with direct dyes, oxidation dyes; dye precursor combinations of a coupler compound and a diazotized compound, or, and/or cationic reactive dyes.

Direct dyes are natural or synthetic; they are uncharged, cationic or anionic, such as acid dyes.

Oxidation dye denotes also for oxidation dye precursors, which are from the group of the developer and coupler compounds. Wherein the coupler compounds denotes also to the addition salts thereof with an acid.

In the context of the present invention the single classes of dyes comprise the dyes defined in the Color Index of the Society of Textile Chemist and Colorist.

Further, in the context of the present invention, combinations comprising of a compound of formula (1) are compositions, formulation and methods.

One preferred embodiment of the present invention is the combination of at least a single compound of formula (1) with a direct dye, which are described in “Dermatology”, edited by Ch. Culnan, H. Maibach, Verlag Marcel Dekker Inc., New York, Basle, 1986, Vol. 7, Ch. Zviak, The Science of Hair Care, chapter 7, pages 248-250, and

in “Europäisches Inventar der Kosmetikrohstoffe”, 1996, published by The European Commission, obtainable in diskette form from the Bundesverband der deutschen Industrie—und Handelsuntemehmen für Arzneimittel, Reformwaren und Körperpflegemittel e.V., Mannheim.

More preferred direct dyes for the combination with at least a single compound of formula (1), especially for semi permanent dyeing, are:

2-Amino-3-nitrophenol, 2-Amino-4-hydroxyethylamino-anisole sulfate, 2-Amino-6-chloro-4-nitrophenol, 2-Chloro-5-nitro-N-hydroxyethylene-p-phenylendiamine, 2-Hydroxyethyl-picramic acid, 2,6-Diamino-3-((pyridine-3yl)-azo)pyridine, 2-Nitro-5-glyceryl-methylaniline, 3-Methylamino-4-nitro-phenoxyethanol, 4-Amino-2-nitrodiphenyleneamine-2′-carboxilic acid, 6-Nitro-1,2,3,4,-tetrahydroquinoxaline, 4-N-Ethyl-1,4-bis(2′-hydroxyethylamino-2-nitrobenzene hydrochloride, 1-Methyl-3-nitro-4-(2′-hydroxyethyl)-aminobenzene, 3-Nitro-p-hydroxyethyl-aminophenol, 4-Amino-3-nitrophenol, 4-Hydroxypropylamine-3-nitrophenol, Hydroxyanthryl-aminopropylmethyl morphlino methosulfat, 4-Nitrophenyl-aminoethylurea, 6-Nitro-p-toluidine, Acid Blue 62, Acid Blue 9, Acid Red 35, Acid Red 87 (Eosin), Acid Violet 43, Acid Yellow 1, Basic Blue 3, Basic Blue 6, Basic Blue 7, Basic Blue 9, Basic Blue 12, Basic Blue 26, Basic Blue 99, Basic Brown 16, Basic Brown 17, Basic Red 2, Basic Red 22, Basic Red 76, Basic Violet 14, Basic Yellow 57, Basic Yellow 9, Disperse Blue 3, Disperse Orange 3, Disperse Red 17, Disperse Violet 1, Disperse Violet 4, Disperse Black 9, Fast Green FCF, HC Blue 2, HC Blue 7, HC Blue 8, HC Blue 12, HC Orange 1, HC Orange 2, HC Red 1, HC Red 10-11, HC Red 13, HC Red 16, HC Red 3, HC Red BN, HC Red 7, HC Violet 1, HC Violet 2, HC Yellow 2, HC Yellow 5, HC Yellow 5, HC Yellow 6, HC Yellow 7, HC Yellow 9, HC Yellow 12, HC Red 8, Hydroxyethyl-2-nitro-p-toluidine, N,N-Bis-(2-Hydroxyethyl)-2-nitro-p-phenylendiamine, HC Violet BS, Picramic Acid, Solvent Green 7.

More preferred are combinations with cationic azo dyes, for example according to GB-A-2 319 776 as well as the oxazine dyes described in DE-A-299 12 327 and mixtures thereof with the other direct dyes mentioned therein.

Preferred direct dyes for the combination with at least a single compound of formula (1) or a combination of at least a single compound of formula (1) and oxidative dyes and oxidization agents, especially for semi permanent dyeing and permanent dyeing, are:

Disperse Violet 4, Picramic acid, N,N′-Bis-(2-Hydroxyethyl)-2-nitro-p-phenylendiamine, HC Yellow No. 5, HC Blue No. 2, HC Yellow No. 2, 2-Chloro-5-nitro-N-hydroxyethyl-p-phenylendiamine, HC Red No. 3, 4-Amino-3-nitrophenol, Basic Blue 99, 2-Hydroxyethyl Picramic acid, HC Yellow No. 6, Hydroxyethyl-2-nitro-p-toluidine, 2-Amino-6-chloro-4-nitrophenol, 4-Hydroxypropylamino-3-nitrophenol, Basic Red 2, HC Red No.16 and HC Blue No. 16.

Especially preferred for a combination with a dye of formula (1) are at least a single direct dye selected from dye of formula (1) of WO 01/66646, especially a direct dye of example 4, and a dye of formula (2) of WO 02/31056, especially a direct dye of example 6, and Basic Yellow 87, and/or Basic Red 51, and Basic Orange 31.

In addition, the present invention concerns the combination of a compound of formula (1) according to the invention with oxidation dyes.

The present invention also describes formulations, which are used for the coloration of keratin fibers, especially human hair.

The formulations are applicable on human hair in different technical forms. The specific technical form may be chosen in view of the envisaged application and/or dye or dye composition. Technical forms of formulation are for example a solution, especially a thickened watery or watery alcoholic solution, a cream, foam, shampoo, powder, a gel, or an emulsion.

Preferred forms of formulations are ready to use compositions or a multi-compartment dyeing device or ‘kit’ or any of the multi-compartment packaging systems with compartments as described for example as described in U.S. Pat. No. 6,190,421, column 2, lines 16 to 31.

It is of advantage to prepare compositions of dyes, which are not stable to reduction, with oxidizing agent free compositions just before the dyeing process.

One preferred embodiment of the present invention concerns the formulation of dyes, especially those of formula (1) in powder form.

The coloring compositions according to the invention may furthermore comprise any active ingredient, additive or adjuvant known for such preparations.

Adjuvants that are suitable for such formulations are in general customary in the field hair-coloring, such as for example surfactants or tensides, solvents, bases, acids, perfumes, polymeric adjuvant, thickeners and light stabilisers.

The coloring composition according to the invention in many cases comprises at least one surfactant. Suitable surfactants are anionic, zwitterionic, ampholytic, non-ionic and cationic surfactants. In many cases, however, it has proved advantageous to select the surfactants from anionic, zwitterionic and non-ionic surfactants.

Anionic surfactants suitable for use in the coloring compositions according to the invention include all anionic surface-active substances that are suitable for use on the human body. Such substances are characterised by an anionic group that imparts water solubility, for example a carboxylate, sulfate, sulfonate or phosphate group, and a lipophilic alkyl group having approximately from 10 to 22 carbon atoms. In addition, glycol or polyglycol ether groups, ester, ether and amide groups and also hydroxy groups may be present in the molecule. The following are examples of suitable anionic surfactants, each in the form of sodium, potassium or ammonium salts or mono-, di- or tri-alkanolammonium salts having 2 or 3 carbon atoms in the alkanol group:
linear fatty acids having from 10 to 22 carbon atoms (soaps),
ether carboxylic acids of formula R—O—(CH₂—CH₂—O)ₓ—CH₂—COOH, in which R is a linear alkyl group having from 10 to 22 carbon atoms and x=0 or from 1 to 16,
acyl sarcosides having from 10 to 18 carbon atoms in the acyl group,
acyl taurides having from 10 to 18 carbon atoms in the acyl group,
acyl isothionates having from 10 to 18 carbon atoms in the acyl group,
sulfosuccinic mono- and di-alkyl esters having from 8 to 18 carbon atoms in the alkyl group and sulfosuccinic monoalkylpolyoxyethyl esters having from 8 to 18 carbon atoms in the alkyl group and from 1 to 6 oxyethyl groups,
linear alkane sulfonates having from 12 to 18 carbon atoms,
linear α-olefin sulfonates having from 12 to 18 carbon atoms,
α-sulfo fatty acid methyl esters of fatty acids having from 12 to 18 carbon atoms,
alkyl sulfates and alkyl polyglycol ether sulfates of formula R′—O(CH₂—CH₂—O)ₓ—SO₃H, in which R′ is a preferably linear alkyl group having from 10 to 18 carbon atoms and x′=O or from 1 to 12,
mixtures of surface-active hydroxysulfonates according to DE-A-3 725 030, especially page 3, lines 40 to 55,
sulfated hydroxyalkylpolyethylene and/or hydroxyalkylenepropylene glycol ethers according to DE-A-3 723 354, especially page 4, lines 42 to 62,
sulfonates of unsaturated fatty acids having from 12 to 24 carbon atoms and from 1 to 6 double bonds according to DE-A-3 926 344, especially page 2, lines 36 to 54,
esters of tartaric acid and citric acid with alcohols which are addition products of approximately from 2 to 15 molecules of ethylene oxide and/or propylene oxide with fatty alcohols having from 8 to 22 carbon atoms, or
anionic surfactants, as described in WO 00/10518, especially page 45, line 11 to page 48, line 3.

Preferred anionic surfactants are alkyl sulfates, alkyl polyglycol ether sulfates and ether carboxylic acids having from 10 to 18 carbon atoms in the alkyl group and up to 12 glycol ether groups in the molecule, and also especially salts of saturated and especially unsaturated C₈-C₂₂carboxylic acids, such as oleic acid, stearic acid, isostearic acid and palmitic acid.

Surface-active compounds that carry at least one quaternary ammonium group and at least one —COO⁽⁻⁾ or —SO₃⁽⁻⁾ group in the molecule are termed zwitterionic surfactants. Zwitterionic surfactants that are especially suitable are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyl-dimethylammonium glycinate, and 2-alkyl-3-carboxymethyl-3-hydroxyethylimidazolines having from 8 to 18 carbon atoms in the alkyl or acyl group and also cocoacylaminoethylhydroxyethylcarboxymethyl glycinate. A preferred zwitterionic surfactant is the fatty acid amide derivative known by the CTFA name cocoamidopropyl betaine.

Ampholytic surfactants are to be understood as meaning surface-active compounds that, in addition to a C₈-C₁₈-alkyl or -acyl group, contain at least one free amino group and at least one —COOH or —SO₃H group in the molecule and are capable of forming internal salts. Examples of suitable ampholytic surfactants include N-alkylglycines, N-alkylpropionic acids, N-alkylaminobutyric acids, N-alkyliminodipropionic acids, N-hydroxyethyl-N-alkylamidopropyl-glycines, N-alkyltaurines, N-alkylsarcosines, 2-alkylaminopropionic acids and alkylaminoacetic acids, each having approximately from 8 to 18 carbon atoms in the alkyl group. Ampholytic surfactants to which special preference is given are N-cocoalkyl-aminopropionate, cocoacylaminoethylaminopropionate and C₁₂-C₁₈acylsarcosine.

Non-ionic surfactants are described in WO 00/10519, especially page 45, line 11 to page 50, line 12.

Non-ionic surfactants contain as the hydrophilic group, for example, a polyol group, a polyalkylene glycol ether group or a combination of polyol and polyglycol ether groups.

Such compounds are, for example:
addition products of from 2 to 30 mol of ethylene oxide and/or from 0 to 5 mol of propylene oxide with linear fatty alcohols having from 8 to 22 carbon atoms, with fatty acids having from 12 to 22 carbon atoms and with alkylphenols having from 8 to 15 carbon atoms in the alkyl group,
C₁₂-C₂₂ fatty acid mono- and di-esters of addition products of from 1 to 30 mol of ethylene oxide with glycerol,
C₈-C₂₂alkyl-mono- and -oligo-glycosides and ethoxylated analogues thereof,
addition products of from 5 to 60 mol of ethylene oxide with castor oil and hydrogenated castor oil,
addition products of ethylene oxide with sorbitan fatty acid esters,
addition products of ethylene oxide with fatty acid alkanolamides.

Examples of cationic surfactants that can be used in the coloring compositions according to the invention are especially quaternary ammonium compounds. Preference is given to ammonium halides, such as alkyltrimethylammonium chlorides, dialkyldimethylammonium chlorides and trialkylmethylammonium chlorides, for example cetyltrimethylammonium chloride, stearyltrimethylammonium chloride, distearyldimethyl-ammonium chloride, lauryldimethylammonium chloride, lauryldimethylbenzylammonium chloride and tricetylmethylammonium chloride. Further cationic surfactants that can be used in accordance with the invention are quaternised protein hydrolysates.

Also suitable in accordance with the invention are cationic silicone oils, such as, for example, the commercially available products Q2-7224 (manufacturer: Dow Corning; a stabilised trimethylsilylamodimethicone), Dow Coming 929 emulsion (comprising a hydroxylamino-modified silicone, which is also referred to as amodimethicone), SM-2059 (manufacturer: General Electric), SLM-55067 (manufacturer: Wacker) and also Abil®-Quat 3270 and 3272 (manufacturer: Th. Goldschmidt; diquaternary polydimethylsiloxanes, quaternium-80), or silicones, as described in WO 00/12057, especially page 45, line 9 to page 55, line 2.

Alkylamidoamines, especially fatty acid amidoamines, such as the stearylamidopropyl-dimethylamine obtainable under the name Tego Amid® 18, are distinguished not only by a good conditioning action but also especially by their good biodegradability.

Quaternary ester compounds, so-called “esterquats”, such as the methyl hydroxyalkyl-dialkoyloxyalkylammonium methosulfates marketed under the trademark Stepantex®, are also very readily biodegradable.

An example of a quaternary sugar derivative that can be used as cationic surfactant is the commercial product Glucquat®100, according to CTFA nomenclature a “lauryl methyl gluceth-10 hydroxypropyl dimonium chloride”.

The alkyl-group-containing compounds used as surfactants may be single substances, but the use of natural raw materials of vegetable or animal origin is generally preferred in the preparation of such substances, with the result that the substance mixtures obtained have different alkyl chain lengths according to the particular starting material used.

The surfactants that are addition products of ethylene and/or propylene oxide with fatty alcohols or derivatives of such addition products may either be products having a “normal” homologue distribution or products having a restricted homologue distribution. “Normal” homologue distribution is to be understood as meaning mixtures of homologues obtained in the reaction of fatty alcohol and alkylene oxide using alkali metals, alkali metal hydroxides or alkali metal alcoholates as catalysts. Restricted homologue distributions, on the other hand, are obtained when, for example, hydrotalcites, alkali metal salts of ether carboxylic acids, alkali metal oxides, hydroxides or alcoholates are used as catalysts. The use of products having restricted homologue distribution may be preferred.

Further preferred active ingredients of formulation according to the present invention, adjuvants and additives are as follows:
non-ionic polymers, for example vinylpyrrolidone/vinyl acrylate copolymers, polyvinyl pyrrolidone and vinylpyrrolidone/vinyl acetate copolymers and polysiloxanes,
cationic polymers, such as quaternised cellulose ethers, polysiloxanes having quaternary groups, dimethyldiallylammonium chloride polymers, copolymers of dimethyldiallylammonium chloride and acrylic acid, as available commercially under the name Merquat® 280 and the use of which in hair coloring is described, for example, in DE-A4 421 031, especially page 2, lines 20 to 49, or EP-A-953 334, especially page 27, line 17 to page 30, line 11, acrylamide/dimethyldiallylammonium chloride copolymers, diethyl-sulfate-quaternised dimethylaminoethyl methacrylate/vinylpyrrolidone copolymers, vinylpyrrolidone/imidazolinium methochloride copolymers,
quaternised polyvinyl alcohol,
zwitterionic and amphoteric polymers, such as, for example, acrylamido-propyl-trimethylammonium chloride/acrylate copolymers and octylacrylamide/methyl methacrylate/tert-butylaminoethyl methacrylate/2-hydroxypropyl methacrylate copolymers,
anionic polymers, such as, for example, polyacrylic acids, crosslinked polyacrylic acids, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert-butyl acrylamide terpolymers,
thickeners, such as agar, guar gum, alginates, xanthan gum, gum arabic, karaya gum, locust bean flour, linseed gums, dextrans, cellulose derivatives, e.g. methyl cellulose, hydroxyalkyl cellulose and carboxymethyl cellulose, starch fractions and derivatives, such amylose, amylopectin and dextrins, clays, e.g. bentonite or fully synthetic hydrocolloids such as, for example, polyvinyl alcohol,
structuring agents, such as glucose and maleic acid,
hair-conditioning compounds, such as phospholipids, for example soya lecithin, egg lecithin, and cephalins, silicone oils, and also conditioning compounds, for example such as those described in DE-A-19 729 080, especially page 2, lines 20 to 49, EP-A-834 303, especially page 2, line 18 to page 3, line 2, or EP-A-312 343, especially page 2, line 59 to page 3, line 11,
protein hydrolysates, especially elastin, collagen, keratin, milk protein, soya protein and wheat protein hydrolysates, condensation products thereof with fatty acids and also quaternised protein hydrolysates,
perfume oils, dimethyl isosorbitol and cydodextrins,
solubilisers, such as ethanol, isopropanol, ethylene glycol, propylene glycol, glycerol and diethylene glycol,
anti-dandruff active ingredients, such as piroctones, olamines and zinc Omadine,
further substances for adjusting the pH value,
active ingredients such as panthenol, pantothenic acid, allantoin, pyrrolidonecarboxylic acids and salts thereof, plant extracts and vitamins,
cholesterol,
light stabilisers and UV absorbers, as described, for example, in EP-A-819 422; especially page 4, lines 34 to 37,
consistency regulators, such as sugar esters, polyol esters or polyol alkyl ethers,
fats and waxes, such as spermaceti, beeswax, montan wax, paraffins, fatty alcohols and fatty acid esters,
fatty alkanolamides,
polyethylene glycols and polypropylene glycols having a molecular weight of from 150 to 50 000, for example such as those described in EP-A-801 942, especially page 3, lines 44 to 55,
complexing agents, such as EDTA, NTA and phosphonic acids,
swelling and penetration substances, such as polyols and polyol ethers, as listed extensively, for example, in EP-A-962 219, especially page 27, lines 18 to 38, for example glycerol, propylene glycol, propylene glycol monoethyl ether, butyl glycol, benzyl alcohol, carbonates, hydrogen carbonates, guanidines, ureas and also primary, secondary and tertiary phosphates, imidazoles, tannins, pyrrole,
opacifiers, such as latex,
pearlising agents, such as ethylene glycol mono- and di-stearate,
propellants, such as propane-butane mixtures, N₂O, dimethyl ether, CO₂ and air, and also
antioxidants,
polyols or polyethers, as described in EP-A-962 219, especially page 27, lines 14 to 38,
thickening polymers, as described in EP-A-970 684, especially page 48, line 16 to page 51, line 4,
sugar-containing polymers, as described in EP-A-970 687, especially page 28, line 17 to page 29, line 23,
quaternary ammonium salts, as described in WO 00/10517, especially page 44, line 16 to page 46, line 23.

The compositions of the present inevtion can also comprise catalysts. Suitable catalysts are metal ions, such as for example Zn²⁺, Cu²⁺, Fe²⁺, F³⁺, Mn²⁺, Mn⁴⁺, Li⁺, Mg²⁺, Ca²⁺ and Al³⁺, preferably Zn²⁺, Cu²⁺ and Mn²⁺.

The metal ions are applicable in any physiological suitable salts form. Preferred salts are acetate, sulfate, halogenide, lactate and tartrate.

Alkalimetalsulfits are in the context of the present invention for example, sodium-, potassium-, lithium-sulfite, and alkalimetaldisulfits are for example sodium-, potassium-, lithium-disulfite, ascorbic acid, tert.-Butylhydrochinon and ammoniumthiolactat.

The use of UV absorbers can effectively protect natural and dyed hair from the damaging rays of the sun and increase the wash fastness of dyed hair.

A preferred embodiment of the present invention concerns the combination of a compound of formula (1) with UV absorbers.

Synergistic effects are observed when UV absorbers are used in combination with antioxidants. Examples of antioxidants that can be used are listened in WO 01/36396 (pages 11-18), U.S. Pat. No. 5,922,310 and U.S. Pat. No. 4,786,493.

Suitable cosmetic preparations may contain usually from 0.05 to 40% by weight, preferably from 0.1 to 20% by weight, based on the total weight of the composition, of one or more UV absorbers.

Of special importance as cosmetic preparations for the hair are the above-mentioned preparations for hair treatment, especially hair-washing preparations in the form of shampoos, hair conditioners, hair-care preparations, e.g. pre-treatment preparations, hair tonics, styling creams, styling gels, pomades, hair rinses, treatment packs, intensive hair treatments, hair-straightening preparations, liquid hair-setting preparations, hair foams and hairsprays. Of special interest are hair-washing preparations in the form of shampoos.

A shampoo has, for example, the following composition: from 0.01 to 5% by weight of a UV absorber according to the invention, 12.0% by weight of sodium laureth-2-sulfate, 4.0% by weight of cocoamidopropyl betaine, 3.0% by weight of sodium chloride, and water ad 100%.

The coloring compositions according to the invention may further comprise antimicrobial agents.

Preferred antimicrobial preservatives and antimicrobial actives used in formulations (in most cases the INCI name of the antimicrobial substances is mentioned):

formaldehyde and paraformaldehyde, hydroxy biphenyls and its salts such as ortho-phenylphenol, zinc pyrithion, chlorobutanol, hydroxy benzoic acids and their salts and esters such as methyl paraben, ethyl paraben, propyl paraben, butyl paraben, dibromo hexamidine and its salts including isothionate (4,4′-hexamethylenedioxy-bis(3-bromo-benzamidine) and 4,4′-hexamethylenedioxy-bis(3-bromo-benzamidinium 2-hydroxyethanesulfonate), mercury, (aceto-O)phenyl (especially phenyl mercuric acetate) and Mercurate(2-),(orthoborate(3-)—O)phenyl, dihydrogene (especially phenyl mercuric borate), 1,3-bis(2-ethylhexyl)-hexahydro-5-methyl-5-pyrimidine (Hexetidin), 5-bromo-5-nitro-1,3-dioxan, 2-bromo-2-nitro-1,3-propandiol, 2,4-dichlorobenzyl alcohol, 3,4,4′trichlorocarbanilide(Trichlorcarban), p-chloro-m-resol, 2,4,4′-trichloro 2-hydroxy diphenylether(triclosan), 4,4′-dichloro 2-hydroxy diphenylether, 4-chloro-3,5-dimethylphenol(Chloroxylenol), imidazolidinyl urea, poly-(hexamethylene biguanide)hydrochloride, 2-phenoxy ethanol(phenoxyethanol), hexamethylene tetramine(Methenamine), 1-(3-chloroallyl)-3,5,7-triaza-1-azonia-adamantanchloride(Quaternium 15), 1-(4-chlorophenyoxy)-1-(1-imidazolyl)3,3-dimethyl-2-butanone(Climbazole), 1,3-bis(hydroxymethyl)-5,5-dimethyl-2,4-imidazolidinedione (DMDM hydantoin), benzyl alcohol, 1,2-dibromo-2,4-dicyano butane, 2,2′methylene-bis(6-bromo-4-chloro phenol)(bromochlorophene), methylchloroisothiazolone, methylisothiazolone, octylisothiazolone, benzylisothiazolone, 2-benzyl-4-chlorophenol(Chlorophenone), chloracetamide, chlorhexidine, chlorhexidine acetate, chlorhexidine gluconate, chlorhexidine hydrochloride, 1-phenoxy-propane-2-ol (phenoxylsopropanol), 4,4-dimethyl-1,3-oxazolidine(dimethyl oxazolidine), diazolidinyl urea, 4,4′-hexamethylenedioxybisbenzamidine and 4,4′-hexamethylenedioxybis(benzamidinium-2-hydroxyethanesulfonate), glutaraldehyde (1,5-pentanedial), 7-ethylbicyclooxazolidine, 3-(4-chlorophenoxy)-1,2-propandiol(chlorophenesin), phenylmethoxymethanol and ((phenylmethoxy)methoxy)-methanol(benzylhemiformal), N-alkyl(C12-C22)trimethyl ammoniumbromide and -chloride(cetrimonium bromide, cetrimonium chloride), benzyl-dimethyl-(4-(2-(4-(1,1,3,3-tetramethylbutyl)-phenoxy)-ethoxy)-ethyl)-ammoniumchloride(benzethonium chloride), Alkyl-(C8-C18)-dimethyl-benzylammonium chloride, -bromide and saccharinate(benzalkonium chloride, benzalkonium bromide, benzalkonium saccharinate), benzoic acid and its salts and esters, propionic acid and its salts, salicylic acid and its salt, sorbic acid and its salts, sodium iodiate, inorganic sulfites and bisulfites such as sodium sulfite, dehydroacetic acid, formic acid, mercurate(1-ethyl)2-mercaptobenzoate(2-)-O,S-,hydrogene(Thiomersal or Thiomerosal), 10-undecylenic acid and its salts, octopirox(piroctone olamine), sodium hydroxy methyl-aminoacetate(sodium hydroxymethylglycinate), 3-iodo-2-propynyl butylcarbamate, 10-undecylenic acid, sulfur.

Combinations with natural antimicrobials or chemically modified natural substances with antimicrobial activities such as chitosans and chitosan derivatives, famesol, plant extracts such as clove oil, blue cypres oil etc. can be also used.

For use on human hair, the dyeing compositions can usually be incorporated into an aqueous cosmetic carrier. Suitable aqueous cosmetic carriers include, for example, creams, sprays, emulsions, gels, powders and also surfactant-containing foaming solutions, e.g. shampoos or other preparations, that are suitable for use on keratin-containing fibers. Such forms of use are described in detail in Research Disclosure 42448 (August 1999).

The dye compound of formula (1), and/or direct dyes may be present in the coloring compositions according to the invention preferably in an amount of from 0.001% to 5%, especially from 0.01% to 1%, based on the total dyeing composition.

The pH value of the ready-to-use dyeing preparations is usually from 2 to 11, preferably from 5 to 10.

The constituents of the aqueous carrier are used in the coloring compositions to the invention in the amounts customary for that purpose; for example emulsifiers may be used in concentrations of from 0.5 to 30% by weight and thickeners in concentrations of from 0.1 to 25% by weight of the total dyeing composition.

The pH-value of the oxidizing agent containing composition is usually about 2 to 7, and in particular about 3 to 6.

An oxidizing agent free composition, which may be added to the coloring compositions according to the invention, comprises a developer compound and a coupler compound and a reduction agent, or

a developer compound or/and optionally a reduction agent, or

a coupler compound and a reduction agent.

Further, an oxidizing agent free composition may additionally comprise a direct dye as for example described in German Patent Application 199 59 479, column 3, line 12 to line 16.

The pH-value of the oxidizing agent free composition is usually about 3 to 11, and in particular about 5 to 10, and most particular about 9 to 10.

In a further embodiment of the present invention concerns processes, especially tinting of hair, for dyeing keratin fibers, in particular human hair, comprising contacting the keratin fiber with at least a compound of formula (1), and then

leaving the fibers to stand, and

then rinsing the fibers.

The process for dyeing is for example described in WO 01/66646 on page 15, line 32 to page 16, line 2.

Usually, the dyeing compositions are usually applied to the hair in an amount of from 50 to 100 g.

This composition is left on the fiber at 15 to 45° C. for 5 to 30 minutes, and in particular for 10 to 20 minutes at 20 to 30° C.

Further preferred a process for dyeing keratin fibers comprises contacting the keratin fibers with at least one direct dye, a base and an oxidizing agent.

The composition comprising at least one direct dye, especially at least a compound of formula (1), a base and an oxidizing agent is prepared by mixing at least one direct dye and a base, and then just before the dyeing of the hair, adding an oxidizing agent.

Alternatively, the oxidizing agent can be applied simultaneously with a composition comprising at least one dye, such as a compound of formula (1) and a base.

The processes of coloring of keratin fibers, especially human hair, with a compound of formula (1) according to the present invention may be combined with other direct dyes and oxidative dyes.

In a preferred embodiment of the present invention the process for dyeing keratin fibers with direct dyes and oxidative dyes, in particular human hair, comprises
a) contacting the keratin fibers with an oxidizing agent, optionally containing at least a compound of formula (1),
b) then contacting the keratin fibers with an oxidizing agent free composition, optionally containing at least a compound of formula (1),

or
a) contacting the keratin fibers with an oxidizing agent free composition, optionally containing at least a compound of formula (1),
b) then contacting the keratin fibers with an oxidizing agent, optionally containing least a compound of formula (1), with the proviso that at least in one of the process steps a) or b) a compound of formula (1) is present.

The process of coloring with a compound of formula (1) according to the present invention may combined with a process for dyeing keratin fibers with direct dyes and oxidative dyes, which comprises

contacting the keratin fibers with least a compound of formula (1),

then contacting the keratin fibers with an oxidizing agent free composition.

Such process is for example described in DE 199 41 450, especially on page 5, lines 50 to 58, and on page 8, line 31 to 46.

Oxidizing agent is usually applied in form of an oxidizing agent containing composition. Oxidizing agent free composition containing at least one coupler compound, at least one developer compound, a base and a reduction agent.

Customary, the oxidizing agent containing composition is evenly applied in a sufficient amount related to the amount of hair, usually with 30 to 200 g.

In general, the oxidizing agent containing composition is left on the fiber at 15 to 45° C. for 0 to 15 minutes, and in particular for 0 to 5 minutes.

Then the oxidizing agent free composition is applied to the hair.

In general, the direct dye and oxidizing agent free composition is left on the fiber at 15 to 50° C. for 5 to 45 minutes, and in particular for 10 to 25 minutes.

The coupler and developer compounds of the oxidizing agent free composition can be applied simultaneously or in succession. Preferred is a simultaneous application.

One preferred embodiment of the process is to wash the hair with shampoo and or a weak acid, such as citric acid or tartrate acid.

The direct dyes, which are stable to reduction can stored together with the oxidizing agent free compositions and are applicable as composition.

It is of advantage to prepare compositions of direct dyes, which are not stable to reduction, with oxidizing agent free compositions just before the dyeing process.

Further, a direct dye and an oxidizing agent free composition can be applied simultaneously or in succession.

A further process for the coloration of keratin fiber with direct dyes and oxidation dyes, which can be used in combination with a compound of formula (1) according to the invention, comprises

mixing at least a compound of formula (1) and optionally at least one coupler compound and at least one developer compound, and an oxidizing agent, which optionally contains at least one further direct dye, and

then contacting the keratin fibers with the mixture as prepared in step a).

A further suitable process for the coloration of keratin fiber with direct dyes and oxidation dyes, which can be used in combination with a compound of formula (1) according to the invention, comprises

mixing at least one autooxidable compound and at least one developer compound and at least one compound of formula (1), and

then contacting the keratin fibers with the mixture prepared above.

The dyes according to the invention are distinguished by brilliant shades. They are suitable for dyeing organic material, such as keratin, wool, leather, silk, paper, cellulose or polyamides, especially keratin-containing fibers, cotton or nylon, and preferably human hair. The dyeing obtained is distinguished by their depth of shade and their good fastness to washing properties, such as, for example, fastness to light, shampooing and rubbing. The stability and storage stability of the dyes according to the invention are excellent. They are accordingly especially suitable for dyeing under oxidizing and reducing conditions. The advantage of the new dyes according to the present invention is their stability against reduction agents e.g. sodium sulfite and ascorbic acid. Therefore, they can be combined with oxidation dyes in one emulsion. Very surprisingly it was found in the present invention that the capped diazotized compounds are applyable on hair in lower amounts than conventional dyes by obtaining the same color strength.

The following Examples serve to illustrate the processes for coloration without limiting the processes thereto. Unless specified otherwise, parts and percentages relate to weight. The amounts of dye specified are relative to the material being colored.

### EXAMPLES A

### Process of Preparation

### EXAMPLE A1

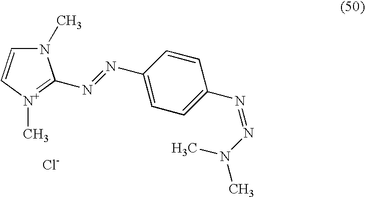

Diazotation

100 ml water and 125 ml concentrate chlorhidric acid (ca. 32%) are given in a well stirred reaction vessel. Then, 0.50 mole (125 g), of the compound of the below formula 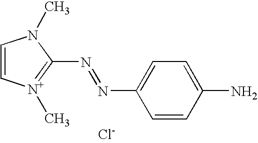
is added. The reaction mixture is cooled to 0° C. At this tempertaure 94.4 g of a solution of 37% sodium nitrite is dropped in during 0.5 to 1 hour to the reaction mixture at such a rate that the temperature is maintained at 0-3° C. The reaction mixture is then stirred for one other hour and controled the excess of nitrite with KI starch paper. If the KI starch is colored more nitrite is added. If the KI starch paper is not colored the reaction is finished and the excess of nitrite destroyed with sulfamic acid. The diazo-solution is ready for coupling and shall stored at temperature of 0° C.

Coupling

70 g of Dimethylamine 40% is dissolved in 200 ml water and then the mixture cooled to 0° C. During 1 hour the diazo solution is slowly dropped into this mixture and the pH adjusted in the range of 9-10 by adding a 36% solution of sodium hydroxide. The reaction temperature is maintained at 0-3° C. by cooling with ice chips, which are dropped into reaction mass. After completing the diazo addition, the suspension is warmed up to room temperature; the pH is adjusted to 10.0 with sodium hydroxide solution. The reaction mixture is then distillated. The humid product is dried at 50-55° C., under vacuum (40-50 mbar). About 233 g dry product is obtained.

The product is characterized by

¹H-NMR Data in deuterated methanol (128 scans)/360 MHz

| | | | | |
|---|---|---|---|---|
| Compound (50) | 8.105 | dd | J=9.0; | 2.03 |
| | | | j=1.8 | |
| | 7.768 | s | | 2.00 |
| | 7.645 | dd | J=9.0 | 1.97 |
| | | | J=1.8 | |
| | 4.165 | s | | 6.06 |
| | 3.655 | s, broad | | 3.01 |
| | 2.715 | s | | 3.5 |

In analogy to the synthesis of compound (50) compounds (57), (60), (61), (62), (63), (64), (65) and (66) are synthesized.

### EXAMPLE A2

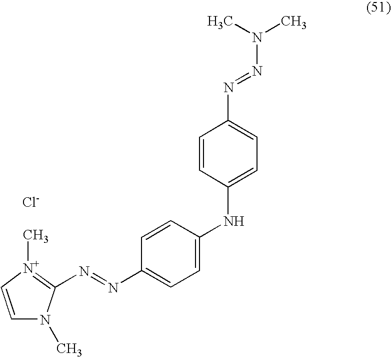

Diazotation

300 ml water and 84 ml concentrate chlorhidric acid (ca. 32%) are given in a well-stirred reaction vessel. Then, 0.30 mole (125 g), of the compound of the below formula 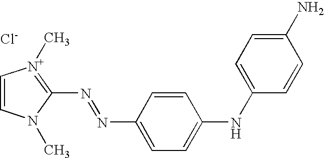
is added. The reaction mixture is cooled to 0° C. At this tempertaure 63 g of a solution of 37% sodium nitrite is dropped in during 0.5 to 1 hour to the reaction mixture at such a rate that the temperature is maintained at 0-3° C. The reaction mixture is then stirred for one other hour. The excess of nitrite is controlled with KI starch paper. If the KI starch is colored, more nitrite is added. If the KI starch paper is not colored, the reaction is finished and the excess of nitrite destroyed with sulfamic acid. The diazo-solution is ready for coupling and shall stored at temperature of 0° C.

Coupling

79 g of Dimethylamine 40% is dissolved in 200 ml water and then the mixture cooled to 0° C. During 1 hour the diazo solution is slowly dropped into this mixture and the pH adjusted in the range of 9-10 by adding a 36% solution of sodium hydroxide. The reaction temperature is maintained at 0-3° C. by cooling with ice chips, which are dropped into reaction mass. After completing the diazo addition, the suspension is warmed up to room temperature; the pH is adjusted to 10.0 with sodium hydroxide solution. The reaction mixture is then distillated. The humid product is dried at 50-55° C., under vacuum (40-50 mbar). About 92 g dry product is obtained.

The product is characterized by ¹H-NMR Data in deuterated methanol (128 scans)/360 MHz

| | | | | |
|---|---|---|---|---|
| Compound (51) | 7.9641 | d | J=9.4 | 2.00 |
| | 7.568 | s | | 2.01 |
| | 7.446 | d | J=8.6 | 1.98 |
| | 7.255 | d | J=8.6 | 1.95 |
| | 7.136 | d | J=9.5 | 2.01 |
| | 4.056 | s | | 6.04 |
| | 3.566 | S, broad | | 4.76 |

### EXAMPLE A3

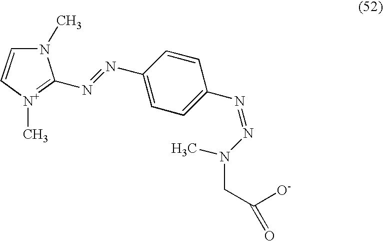

Diazotation

100 ml water and 125 ml concentrate chlorhidric acid (ca. 32%) are given in a well-stirred reaction vessel. Then, 0.50 mole of the compound of the below formula 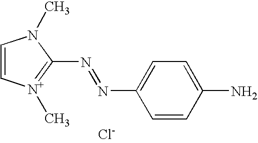
is added. The reaction mixture is cooled to 0° C. At this tempertaure 94.4 g of a solution of 37% sodium nitrite is dropped in during 0.5 to 1 hour to the reaction mixture at such a rate that the temperature is maintained at 0-3° C. The reaction mixture is then stirred for one other hour. The excess of nitrite is controled with KI starch paper. If the KI starch is colored, more nitrite is added. If the KI starch paper is not colored, the reaction is finished and the excess of nitrite destroyed with sulfamic acid. The diazo-solution is ready for coupling and shall stored at temperature of 0° C.

Coupling

55 g sarcosine is dissolved in 100 ml water and then the mixture cooled to 0 ° C. During 1 hour the diazo solution is slowly dropped into this mixture and the pH adjusted in the range of 9-10 by adding a 36% solution of sodium hydroxide. The reaction temperature is maintained at 0-3° C. by cooling with ice chips, which are dropped into reaction mass. After completing the diazo addition, the suspension is warmed up to room temperature; the pH is adjusted to 10.0 with sodium hydroxide solution. The reaction mixture is then distillated completely dry and extracted with 2*500 ml water free ethanol. The sodium chloride salt is filtrated off. The filtrate is concentrated by distillation. By cooling at 4-5° C., the product precipitates. The suspension is then filtrated, washed twice with 50 ml of ethanol. The humid product is dried at 50-55° C., under vacuum (40-50 mbar). About 160 g dry product is obtained.

The product a mixture of two components and is characterized by

¹H-NMR Data in deuterated methanol (128 scans)/360 MHz

| | | | | |
|---|---|---|---|---|
| Compound (52) | 8.028 | dd | J=9.0 | 2.0 |
| | | | j=1.4 | |
| | 7.758 | s | | 2.0 |
| | 7.601 | | J=8.8 | 1.98 |
| | | | J=1.4 | |
| | 4.482 | s | | 2.06 |
| | 4.404 | | | |
| | 4.116 | s | | 5.24 |
| | 3.396 | s | | 3.01 |
| | 3.375 | | | |

In analogy to the synthesis of compound (52) compound (56) is synthesized.

### EXAMPLE A4

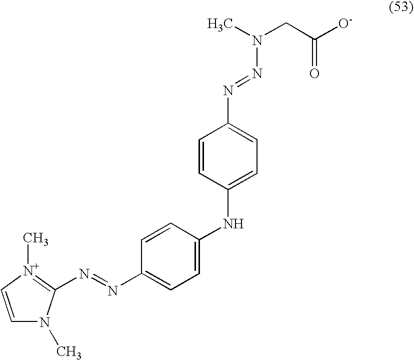

Diazotation

300 ml water and 84 ml concentrate chlorhidric acid (ca. 32%) are given in a well-stirred reaction vessel. Then, 0.30 mole (113 g), of the compound of the below formula 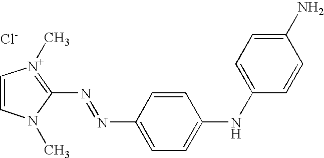
is added. The reaction mixture is cooled to 0° C. At this tempertaure 63 g of a solution of 37% sodium nitrite is dropped in during 0.5 to 1 hour to the reaction mixture at such a rate that the temperature is maintained at 0-3° C. The reaction mixture is then stirred for one other hour. The excess of nitrite is controlled with KI starch paper. If the KI starch is colored, more nitrite is added. If the KI starch paper is not colored, the reaction is finished and the excess of nitrite destroyed with sulfamic acid. The diazo-solution is ready for coupling and shall stored at temperature of 0° C.

Coupling

36 g sarcosine is dissolved in 200 ml water and then the mixture cooled to 0° C. During 1 hour the diazo solution is slowly dropped into this mixture and the pH adjusted in the range of 9-10 by adding a 36% solution of sodium hydroxide. The reaction temperature is maintained at 0-3° C. by cooling with ice chips, which are dropped into reaction mass.

After completing the diazo addition, the suspension is warmed up to room temperature; the pH is adjusted to 10.0 with sodium hydroxide solution. The reaction mixture is then filtrated, washed twice with 50 ml of water. The humid product is dried at 50-55° C., under vacuum (40-50 mbar). About 156 g dry product is obtained.

The product is characterized by

¹H-NMR Data in deuterated methanol (128 scans)/360 MHz

| | | | | |
|---|---|---|---|---|
| Compound (53) | 7.921 | d | J=9.0 | 2.05 |
| | 7.512 | s | | 2.0 |
| | 7.407 | d | J=8.6 | 2.11 |
| | 7.193 | d | J=8.1 | 2.20 |
| | 7.091 | d | J=9.0 | 2.02 |
| | 4.352 | S, broad | | 1.96 |
| | 4.017 | S | | 6.18 |
| | 3.278 | S, broad | | 3.01 |

### EXAMPLE A5

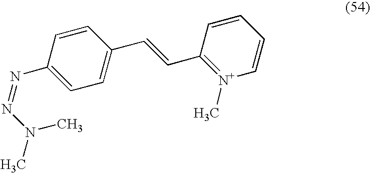

Diazotation

300 ml water and 84 ml concentrate chlorhidric acid (ca. 32%) are given in a well-stirred reaction vessel. Then, 0.30 mole, of the compound of the below formula 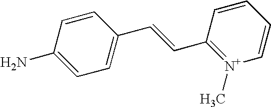
is added. The reaction mixture is cooled to 0° C. At this tempertaure 63 g of a solution of 37% sodium nitrite is dropped in during 0.5 to 1 hour to the reaction mixture at such a rate that the temperature is maintained at 0-3° C. The reaction mixture is then stirred for one other hour. The excess of nitrite is controlled with KI starch paper. If the KI starch is colored, more nitrite is added. If the KI starch paper is not colored, the reaction is finished and the excess of nitrite destroyed with sulfamic acid. The diazo-solution is ready for coupling and shall stored at temperature of 0° C.

Coupling

79 g of dimethylamine 40% is dissolved in 200 ml water and then the mixture cooled to 0° C. During 1 hour the diazo solution is slowly dropped into this mixture and the pH adjusted in the range of 9-10 by adding a 36% solution of sodium hydroxide. The reaction temperature is maintained at 0-3° C. by cooling with ice chips, which are dropped into reaction mass.

After completing the diazo addition, the suspension is warmed up to room temperature; the pH is adjusted to 10.0 with sodium hydroxide solution. The reaction mixture is then filtrated, and washed twice with 50 ml of water. The humid product is dried at 50-55° C., under vacuum (40-50 mbar). About 156 g dry product is obtained.

The product is characterized by

¹H-NMR Data in deuterated methanol (128 scans)/360 MHz

| | | | | |
|---|---|---|---|---|
| Compound (54) | 8.762 | d | J = 6.3 | 1.05 |
| | 8.434 | s | | 2.0 |
| | 8.423 | s | | |
| | 7.856 | d | J = 16 | |
| | 7.78 | d | J = 8.5 | 3.91 |
| | 7.091 | t | J = 6.3 | |
| | 7.48 | d | J = 8.5 | 1.85 |
| | 7.45 | d | J = 16 | 1 |
| | 4.394 | s | | 2.96 |
| | 3.41 | s, broad | | 6.18 |

In analogy to the synthesis of compound (54) compound (58) is synthesized.

### EXAMPLE A6

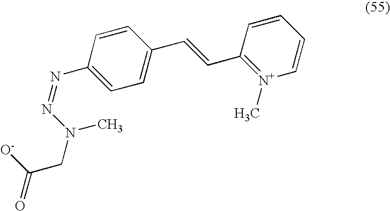

Diazotation

300 ml water and 84 ml concentrate chlorhidric acid (ca. 32%) are given in a well-stirred reaction vessel. Then, 0.30 mole, of the compound of the below formula 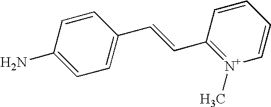
is added. The reaction mixture is cooled to 0° C. At this tempertaure 63 g of a solution of 37% sodium nitrite is dropped during 0.5 to 1 hour to the reaction mixture at such a rate that the temperature is maintained at 0-3° C. The reaction mixture is then stirred for one other hour. The excess of nitrite is controlled with KI starch paper. If the KI starch is colored, more nitrite is added. If the KI starch paper is not colored, the reaction is finished and the excess of nitrite destroyed with sulfamic acid. The diazo-solution is ready for coupling and shall stored at temperature of 0° C.

Coupling

36 g of sarcosine is dissolved in 200 ml water and then the mixture cooled to 0° C. During 1 hour the diazo solution is slowly dropped into this mixture and the pH adjusted in the range of 9-10 by adding a 36% solution of sodium hydroxide. The reaction temperature is maintained at 0-3° C. by cooling with ice chips, which are dropped into reaction mass.

After completing the diazo addition, the suspension is warmed up to room temperature; the pH is adjusted to 10.0 with sodium hydroxide solution. The reaction mixture is then filtrated, and washed twice with 50 ml of water. The humid product is dried at 50-55° C., under vacuum ( 40-50 mbar). About 156 g dry product is obtained.

The product is characterized by

¹H-NMR Data in deuterated methanol (128 scans)/360 MHz

| | | | | |
|---|---|---|---|---|
| Compound (55) | 8.721 | d | J = 6.0 | 1.00 |
| | 8.35 | m | | 2.00 |
| | 7.70 | m | | 4.02 |
| | 7.293 | d | J = 8.1 | 1.80 |
| | 7.281 | d | J = 16.0 | 1.02 |
| | 4.414 | s, | | 1.96 |
| | 4.281 | s | | 3.10 |
| | 3.319 | s | | 3.01 |

In analogy to the synthesis of compound (55) compound (59) is synthesized.

### EXAMPLE B

### Application

Coloring Method A:

A strand of bleached human hair is immersed, for 30 minutes at room temperature, in an aqueous solution containing 0.02M capped diazonium compound and 0.02M coupling component, which has been adjusted to pH 10.0 using sodium carbonate, ammonia or NaOH. The strand is removed, excess solution is wiped off and the strand is immersed for 5 minutes in a pH 3 buffer solution containing 4% sodium citrate and 2% citric acid. The strand is then thoroughly rinsed using water and, where appropriate, a shampoo solution and is dried. Hair colored in the shades mentioned is obtained, with outstanding fastness properties, especially fastness to washing properties.

Coloring Method B:

A strand of bleached human hair is immersed, for 30 minutes at room temperature, in an aqueous solution that contains 0.02M capped diazonium compound, 0.2M coupling component and 0.2M of hydrogen peroxide (6%) and that has been adjusted to a pH in the range pH 9.8-10 using sodium carbonate, ammonia or NaOH. After contact for 5-30 minutes, there is applied to the strand, without its being rinsed, an amount, corresponding to the weight of triazene and coupling component originally used, of a mixture comprising 12.5% strength aqueous citric acid gel, which contains 0.1% by weight of a cationic dye selected from the group of the cationic dyes as disclosed in WO 95/01772 and in WO 01/66646. The strand is then combed through thoroughly, a pH of about 7 being obtained. After contact for 15 minutes, the treated strand is treated again with the above mixture comprising 12.5% strength citric acid gel and 0.1% by weight of a cationic dye selected from the group of the cationic dyes as disdosed in WO 95/01772 and in WO 01/66646 at pH 4 for 5 minutes, rinsed thoroughly with water and then dried. Hair is obtained with outstanding fastness properties, especially fastness to washing and fastness to shampooing properties.

Coloring Method C:

A strand of bleached human hair is immersed, for 30 minutes at room temperature, in an aqueous solution that contains 0.02M capped diazonium compound, 0.02M coupling component and 0.2 mol of hydrogen peroxide (6%) and that has been adjusted to a pH in the range pH 9.8-10 using sodium carbonate, ammonia or NaOH. After contact for 5-30 minutes, there is applied to the hair, without its being rinsed, an amount, corresponding to the weight of triazene and coupling component originally used, of a 12.5% strength aqueous citric acid gel containing 0.1% by weight of a cationic dye selected from the group of the cationic dyes as disclosed in WO 95/01772 and in WO 01/66646 and 4% sodium citrate; the hair is combed through thoroughly, a pH of about 3 being obtained. Then, after a contact time of 5-30 minutes, the hair is rinsed thoroughly with water and dried. Hair is obtained with outstanding fastness properties, especially good fastening to washing properties.

Coloring Method D:

A strand of bleached human hair is immersed, for 30 minutes at room temperature, in an aqueous solution containing 0.02M capped diazonium compound, 0.02M coupling component, 0.2 mol of hydrogen peroxide (6%) and from 0.1 to 1% by weight, based on the weight of the triazene and coupling component, of a cationic dye selected from the group of the cationic dyes as described in WO 95/01772 and in WO 01/66646. The strand is then adjusted to a pH in the range pH 9.8-10 using sodium carbonate, ammonia or NaOH. After contact for 5-30 minutes, there is applied to the hair, without its being rinsed, an amount, corresponding to the weight of triazene and coupling component originally used, of a 12.5% strength aqueous citric acid gel and 4% sodium citrate and the hair is combed through thoroughly, a pH of about 3 being obtained. Then, after a contact time of 5-30 minutes, the hair is rinsed thoroughly with water and dried. Hair is obtained with outstanding fastness properties, especially good fastness to washing properties.

### EXAMPLE B1

### Application

A strongly alkaline 10% solution of a non-ionic surfactant (Plantaren 2000, Henkel) is adjusted to pH 9.5 using citric acid. 0.01% of the dye of formula (50) as given in example A1 is dissolved therein and a strand of human hair, bleached white, is treated with the dye solution at room temperature. After only a short period of time, the strand has been dyed a bluish-red shade, which is still very intensive even shampooing ten times. The dye also has a strong affinity to undamaged hair. In that case, too, the wash fastness is very good. The light fastness on damaged and undamaged hair is excellent. The perm fastness is on un- and damaged hair very good as well.

### EXAMPLE B2

A 10% solution of a non-ionic surfactant (Plantaren 2000, Henkel) is adjusted to pH 5.5 using citric acid. 0.01% of the dye of formula (51) as given in example A2 is dissolved therein and a strand of middle blonde undamaged human hair is treated with the dye solution at room temperature. After only a short period of time, the strand has been dyed in a bluish shade, which has a good wash, perm and light fastness.

### EXAMPLE B3

A dye emulsion, containing

0.01% of the dye according to the invention of formula (52) as given in example A3, and

3.5% cetearyl alcohol

1.0% ceteareth 80

0.5% glyceryl mono-di-stearate

3.0% stearamide DEA

1.0% stearamphopropyl sulfonate

0.5% polyquarternium-6 and

water ad 100%

is applied for 30 minutes, at room temperature, to bleached human hair, and rinsed. The result is a very attractive vibrant red dyeing with good fastnesses.

### EXAMPLE B4

A dye emulsion, containing 0.1% of the dye of formula (53) as given in example A4, pH=9.8

| | |
|---|---|
| Cetylstearylalcohol | 11.00 |
| Oleth-5 | 5.0 |
| Oleic acid | 2.5 |
| Stearic acid monoethanolamide | 2.5 |
| Coco fatty acid monoethanolamide | 2.5 |
| Sodium laurylsuphate | 1.7 |
| 1,2-Propanediol | 1.0 |
| Ammoniumchloride | 0.5 |
| EDTA, Tetrasodiumsalt | 0.2 |
| Perfume | 0.4 |
| Cornproteinhydrolysate | 0.2 |
| Silica | 0.1 |

is mixed with the same weight of 6% hydrogen peroxide solution and the mixture is immediately applied to a tress of brown hair. After 30 minutes the tress is rinsed, shampooed, rinsed and dried.

### EXAMPLE B5

A dye emulsion, containing 0.05% of the dye of formula (54) as given in example A5, pH=9.8

| | |
|---|---|
| Cetylstearylalcohol | 11.00 |
| Oleth-5 | 5.0 |
| Oleic acid | 2.5 |
| Stearic acid monoethanolamide | 2.5 |
| Coco fatty acid monoethanolamide | 2.5 |
| Sodium laurylsuphate | 1.7 |
| Sodiumsulphite | 1.0 |
| Ascorbic acid | 0.5 |
| 1,2-Propanediol | 1.0 |
| Ammoniumchloride | 0.5 |
| EDTA, Tetrasodiumsalt | 0.2 |
| Perfume | 0.4 |
| Cornproteinhydrolysate | 0.2 |
| Silica | 0.1 |
| Toluene-2,5-Diamine Sulfate | 0.07 |
| Resorcinol | 0.02 |
| 2-Amino-6-Chloro-4-Nitrophenol | 0.01 |
| 4-Amino-m-Cresol | 0.03 |
| 2-Amino-3-Hydroxypyridine | 0.001 |

is mixed with the same weight of 6% hydrogen peroxide solution and the mixture is immediately applied to a tress of brown hair. After 30 minutes the tress is rinsed, shampooed, rinsed and dried.

The color result is a very brilliant violett shade.

### EXAMPLE B6

A strongly alkaline 10% solution of a non-ionic surfactant (Plantaren 2000, Henkel) is adjusted to pH 9.5 using citric acid. 0.01% of the dye of formula (50) as given in example A1 and 0.1% of the dye formula 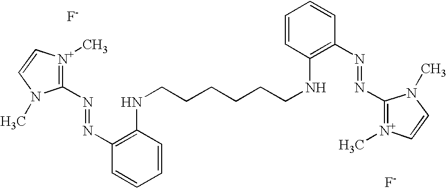
are dissolved therein and a strand of middle blonde undamaged human hair is treated with the dye solution at room temperature. After only 10 minutes, the strand has has a good wash, perm and light fastness.

### EXAMPLE B7

A strongly alkaline 10% solution of a non-ionic surfactant (Plantaren 2000, Henkel) is adjusted to pH 9.5 using citric acid. 0.01% of the dye (60) and 0.1% of the dye formula (50) as given in example A1 are dissolved therein and a strand of dark blonde undamaged human hair is treated with the dye solution at room temperature. After 20 minutes, the strand has been dyed a intensive aubergine shade, which has a good wash, perm and light fastness.

### EXAMPLE B8

First Step:

A strand bleached blond hair is treated with a composition comprising 5 g of 6% by weight hydrogenperoxide solution and 5 g a composition comprising an oxidation base with a pH value of 9.8 as given below:

Composition comprising an oxidation base with a pH value of 9.8

| | |
|---|---|
| 10.0% by weight olic acid | |
| Toluene-2,5-diamine sulfate | 0.07 |
| Resorcinol | 0.02 |
| 2-Amino-6-chloro-4-nitrophenol | 0.01 |
| 4-Amino-m-cresol | 0.03 |
| 2-Amino-3-hydroxypyridine | 0.001 |
| Sodium sulfite | 1.0 |
| Ascorbinic acid | 0.5 |
| Water | ad 100 |

### EXAMPLE B8a

### Second Step

After 15 minutes, the pH of the hair is adjusted to pH 5 by addition of citric acid. Then, 5 g of a 12,5% citric acid gel, comprising the 0.1% by weight of dye of formula (51) of example A2 according to the present application, is applied on the hair and combed, so that the hair has a pH of 7. After 15 minutes the hair is washed with water, rinsed and dried. The strand has been dyed in an intensive shade, which has a good wash and light fastness.

### EXAMPLE B8a

### Second Step

After 15 minutes, without rinsing, blond hair is treated with a composition comprising 5 g of 6% by weight hydrogenperoxide solution and 5 g a composition comprising an oxidation base with a pH value of 5 as given below:

Composition comprising an oxidation base with a pH value of 5 (adjusted with citric acid)

| | |
|---|---|
| 10.0% by weight Olic acid | |
| Toluene-2,5-diamine sulfate | 0.07 |
| Resorcinol | 0.02 |
| 2-Amino-6-chloro-4-nitrophenol | 0.01 |
| 4-Amino-m-cresol | 0.03 |
| 2-Amino-3-hydroxypyridine | 0.001 |
| Sodium sulfite | 1.0 |
| Ascorbinic acid | 0.5 |
| Water | ad 100 |

and 5 g of a 12.5% citric acid gel, comprising the 0.01% by weight of dye of formula (52) of example A3 according to the present application,

The hair is combed, so that the hair has a pH of 7. After 15 minutes the hair is washed with water, rinsed and dried. The strand has been dyed in an intensive shade, which has a good wash and light fastness.

### EXAMPLE B8c

### Second Step

After 15 minutes the hair is treated with 5 g composition comprising an oxidation base with a pH value of 9.8 as given below:

Composition comprising an oxidation base with a pH value of 9.8

| | |
|---|---|
| 10.0% by weight Olic acid | |
| Toluene-2,5-diamine sulfate | 0.07 |
| Resorcinol | 0.02 |
| 2-Amino-6-chloro-4-nitrophenol | 0.01 |
| 4-Amino-m-cresol | 0.03 |
| 2-Amino-3-hydroxypyridine | 0.001 |
| Sodium sulfite | 1.0 |
| Ascorbinic acid | 0.5 |
| Water | ad 100 |

### EXAMPLE B8a

### Second Step

After 15 minutes, the pH of the hair is adjusted to pH 5 by addition of citric acid. Then, 5 g of a 12,5% citric acid gel, comprising the 0.1% by weight of dye of formula (61) according to the present application, is applied on the hair and combed, so that the hair has a pH of 7. After 15 minutes the hair is washed with water, rinsed and dried. The strand has been dyed in an intensive shade, which has a good wash and light fastness.

### EXAMPLE B8a

### Second Step

After 15 minutes, without rinsing, blond hair is treated by a comb with a composition comprising 5 g of 6% by weight hydrogenperoxide solution and 5 g of a composition comprising an oxidation base with a pH value of 9.8 as given below:

Composition comprising an oxidation base with a pH value of 9.8

| | |
|---|---|
| 10.0% by weight Olic acid | |
| Toluene-2,5-diamine sulfate | 0.07 |
| Resorcinol | 0.02 |
| 2-Amino-6-chloro-4-nitrophenol | 0.01 |
| 4-Amino-m-cresol | 0.03 |
| 2-Amino-3-hydroxypyridine | 0.001 |
| Sodium sulfite | 1.0 |
| Ascorbinic acid | 0.5 |
| Water | ad 100 |

and a solution comprising 1% by weight of a dye of formula (62) according to the present application with a pH of 9.8. After 15 minutes the hair is washed with water, rinsed and dried. The strand has been dyed in an intensive shade, which has a good wash and light fastness.

### EXAMPLE B9

A strand of bleached human hair is treated with a mixture of equal parts by weight—5 g in each case—of 6% hydrogen peroxide solution and of composition A.

Composition A:

| | |
|---|---|
| Cetyl stearyl alcohol | 11.00 |
| Oleth-5 | 5.0 |
| Oleic acid | 2.5 |
| Stearic acid monoethanolamide | 2.5 |
| Coconut fatty acid monoethanolamide | 2.5 |
| Sodium lauryl sulfate | 1.7 |
| 1,2-propanediol | 1.0 |
| ammonium chloride | 0.5 |
| EDTA, tetrasodium salt | 0.2 |
| Perfume | 0.4 |
| Wheat protein hydrolysate | 0.2 |
| Silica | 0.1 |
| 2,5-Diaminotoluene sulfate | 0.7 |
| 4-Amino-2-hydroxytoluene | 0.5 |
| 2,5,6-Triamino-4-hydroxypyrimidine sulfate | 0.2 |
| Sodium sulfite | 1.0 |
| Ascorbic acid | 0.5 |
| Compound (50) of example A1 | 9.32 |
| Composition A: pH | 9.8 |
| Water | ad 100 |

After contact for 15 minutes at room temperature, about 22° C., 10 g of a mixture of a 12.5% strength aqueous citric acid gel containing 0.1% by weight of a violet dye of the following formula 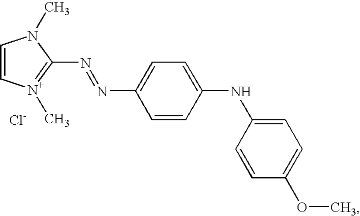
which is prepared analogously to WO 01/66646, Example 4, is applied to the strand. The strand is then combed through, whereupon a pH of about 7 is achieved. After contact for a further 15 minutes, the strand is again treated with 10 g of the above mixture of citric acid gel and violet dye, whereupon a pH of about 4 is achieved. The mixture is allowed to act for 5 minutes at pH 4 and the strand is then washed with water and shampoo and then again with water. The strand is then dried.

A strong, intense, striking coloration having good fastness to washing and fastness to rubbing properties is obtained.

### EXAMPLE B10

A strand of medium-blond human hair is coloured with a mixture of equal parts by weight—5 g in each case—of 6% hydrogen peroxide solution and of composition A according to Example B9.

The mixture is allowed to act on the strand for 30 minutes at room temperature, about 22° C. 10 g of a mixture of a 2% strength aqueous citric acid gel containing 0.1% by weight of a violet dye according to WO 01/66646, example 4, and 4% sodium citrate, are then applied to the strand. The strand is then combed through, whereupon a pH of about 3 is achieved. After contact for 5 minutes, the strand is thoroughly rinsed and then dried.

A strong, intense, striking coloration having good fastness to washing and fastness to rubbing properties is obtained.

### EXAMPLE B11

A strand of bleached human hair is coloured with a mixture of equal parts by weight—-5 g in each case—of 6% hydrogen peroxide solution and of composition B.

Composition B:

| | |
|---|---|
| Cetyl stearyl alcohol | 11.00 |
| Oleth-5 | 5.0 |
| Oleic acid | 2.5 |
| Stearic acid monoethanolamide | 2.5 |
| Coconut fatty acid monoethanolamide | 2.5 |
| Sodium lauryl sulfate | 1.7 |
| 1,2-Propanediol | 1.0 |
| Ammonium chloride | 0.5 |
| EDTA, tetrasodium salt | 0.2 |
| Perfume | 0.4 |
| Wheat protein hydrolysate | 0.2 |
| Silica | 0.1 |
| Compound (52) of example A3 | 9.32 |
| Composition B: pH | 9.8 |
| Water | ad 100 |

The mixture is allowed to act on the strand for 30 minutes at about 22° C. 10 g of a mixture of a 2% strength aqueous citric acid gel containing 0.1% by weight of a violet dye according to WO 01/66646, Example 4, and 4% sodium citrate, are then applied to the strand. The strand is then combed through, whereupon a pH of about 3 is achieved. After contact for 5 minutes, the strand is thoroughly rinsed and then dried.

A strong, intense, striking coloration having good fastness to washing and fastness to rubbing properties is obtained.

### EXAMPLE B12

A strand of blond undamaged human hair is coloured with a mixture of equal parts by weight—5 g in each case—of 6% hydrogen peroxide solution and of composition C.

Composition C:

| | |
|---|---|
| Cetyl stearyl alcohol | 11.00 |
| Oleth-5 | 5.0 |
| Oleic acid | 2.5 |
| Stearic acid monoethanolamide | 2.5 |
| Coconut fatty acid monoethanolamide | 2.5 |
| Sodium lauryl sulfate | 1.7 |
| 1,2-Propanediol | 1.0 |
| Ammonium chloride | 0.5 |
| EDTA, tetrasodium salt | 0.2 |
| Perfume | 0.4 |
| Wheat protein hydrolysate | 0.2 |
| Silica | 0.1 |
| Violet dye of WO 01/66646, Example 4, | 0.2 |
| Compound (52) of example A3 | 9.32 |
| Composition C: pH | 9.8 |
| Water | ad 100 |

The mixture is allowed to act on the strand for 30 minutes at about 22° C. 10 g of a mixture of a 2% strength aqueous citric acid gel containing 0.1% by weight of a violet dye according to WO 01/66646, Example 4, and 4% sodium citrate, are then applied to the strand; the latter is then combed through, whereupon a pH of about 3 is achieved. After contact for 5 minutes, the strand is thoroughly rinsed and then dried.

A strong, intense, striking violet coloration having good fastness to washing and fastness to rubbing properties is obtained.

### EXAMPLE B13

A strand of blond undamaged human hair is coloured with 10 g of composition D.

Composition D:

| | |
|---|---|
| Cetyl stearyl alcohol | 11.00 |
| Oleth-5 | 5.0 |
| Oleic acid | 2.5 |
| Stearic acid monoethanolamide | 2.5 |
| Coconut fatty acid monoethanolamide | 2.5 |
| Sodium lauryl sulfate | 1.7 |
| 1,2-Propanediol | 1.0 |
| Ammonium chloride | 0.5 |
| EDTA, Tetrasodium salt | 0.2 |
| Perfume | 0.4 |
| Wheat protein hydrolysate | 0.2 |
| Silica | 0.1 |
| Violet dye of WO 01/66646, Example 4, | 0.2 |
| Compound (52) of example A3 | 4.66 |
| 4-Amino-2-hydroxytoluene | 5.76 |
| composition D: pH | 10 |
| Water | ad 100 |

The mixture is allowed to act on the strand for 30 minutes at about 22° C. 10 g of a mixture of a 2% strength aqueous citric acid gel containing 4% sodium citrate are then applied to the strand; the latter is then combed through, whereupon a pH of about 3 is achieved. After contact for 5 minutes, the strand is thoroughly rinsed and then dried. A strong, intense, striking coloration having good fastness to washing and fastness to rubbing properties is obtained.

### EXAMPLE B14

A strand of blond undamaged human hair is coloured with 10 g of composition E.

Composition E

| | |
|---|---|
| Cetyl stearyl alcohol | 11.00 |
| Oleth-5 | 5.0 |
| Oleic acid | 2.5 |
| Stearic acid monoethanolamide | 2.5 |
| Coconut fatty acid monoethanolamide | 2.5 |
| Sodium lauryl sulfate | 1.7 |
| 1,2-Propanediol | 1.0 |
| Ammonium chloride | 0.5 |
| EDTA, Tetrasodium salt | 0.2 |
| Perfume | 0.4 |
| Wheat protein hydrolysate | 0.2 |
| Silica | 0.1 |
| Compound (54) of example A5 | 4.66 |
| 4-Amino-2-hydroxytoluene | 5.76 |
| Composition E: pH | 10 |
| Water | ad 100 |

The mixture is allowed to act on the strand for 30 minutes at about 22° C. Then 10 g of a mixture of a 2% strength aqueous citric acid gel containing 0.1% by weight of a red dye of the following formula 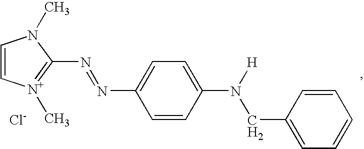
which can be prepared, for example, as described in WO 01/11708, according to example 6, for compound of formula 106,

and 4% sodium citrate, are applied to the strand and then combed through, whereupon a pH of about 3 is achieved. After contact for 5 minutes, the strand is thoroughly rinsed and then dried.

A strong, intense, striking coloration having good fastness to washing and fastness to rubbing properties is obtained.

### EXAMPLE B15

A strand of blond undamaged human hair is coloured with 10 g of composition F.

Composition F

| | |
|---|---|
| Cetyl stearyl alcohol | 11.00 |
| Oeth-5 | 5.0 |
| OLeic acid | 2.5 |
| STearic acid monoethanolamide | 2.5 |
| Coconut fatty acid monoethanolamide | 2.5 |
| Sodium lauryl sulfate | 1.7 |
| 1,2-Propanediol | 1.0 |
| Ammonium chloride | 0.5 |
| EDTA, tetrasodium salt | 0.2 |
| Perfume | 0.4 |
| Wheat protein hydrolysate | 0.2 |
| Silica | 0.1 |
| Compound of example 54 of Example A5 | 5.3 |
| Toluene-2,5-diamine Sulfate | 5.5 |
| Composition F: pH | 10 |
| Water | ad 100 |

After contact for 30 minutes, without being washed out, a dye mixture known from U.S. Pat. No. 6,248,314 and having the following composition:

| | |
|---|---|
| Black Color No. 401 | 0.1 |
| Purple Color 401 | 0.05 |
| Orange Color No. 205 | 0.1 |
| Benzyl alcohol | 2.0 |
| Ethylene carbonate | 10 |
| Propylene carbonate | 15 |
| Ethanol | 10 |
| Lactic acid | 3.5 |
| Sodium carbonate solution | of pH 2.9 |
| hydroxyethyl cellulose | 1.5 |
| Water | ad 100 |

is applied to the hair. The hair is then combed through thoroughly, whereupon its pH becomes about 3. Then, after a contact period of 15 minutes, the hair is rinsed thoroughly with water and dried.

A strong, intense, striking coloration having good fastness to washing and fastness to rubbing properties is obtained.

### EXAMPLE B16

A strand of blond undamaged human hair is coloured with 10 g of a composition comprising compositions A.

Compositions

| | A | C |
|---|---|---|
| Cetyl stearyl alcohol | 11.00 | 11.00 |
| Oleth-5 | 5.0 | 5.0 |
| Oleic acid | 2.5 | 2.5 |
| Stearic acid monoethanolamide | 2.5 | 2.5 |
| Coconut fatty acid monoethanolamide | 2.5 | 2.5 |
| Sodium lauryl sulfate | 1.7 | 1.7 |
| 1,2-Propanediol | 1.0 | 1.0 |
| Ammonium chloride | 0.5 | 0.5 |
| EDTA, tetrasodium salt | 0.2 | 0.2 |
| Perfume | 0.4 | 0.4 |
| Wheat protein hydrolysate | 0.2 | 0.2 |
| Silica | 0.1 | 0.1 |
| 2,5-Diaminotoluene sulfate | | 0.7 |
| 4-Amino-2-hydroxytoluene | | 0.5 |
| 2,5,6-Triamino-4-hydroxypyrimidine | | 0.2 |
| sulfate | | |
| Sodium sulfite | | 1.0 |
| Ascorbic acid | | 0.5 |
| Compound (55) of example A4 . . . | 9.32 | |
| Composition: pH | 10 | 10 |
| Water | ad 100 | ad 100 |

The colouring mixture is allowed to act on the hair for 30 minutes at about 22° C. 10 g of a 2% strength aqueous citric acid gel are then applied to the strand. After contact for 5 minutes, the strand is rinsed thoroughly, shampooed and then dried.

A strong, intense, striking coloration having good fastness to washing and fastness to rubbing properties is obtained.

### EXAMPLE B17

A strand of blond undamaged human hair is coloured with a mixture of 15 g of 6% hydrogen peroxide solution and a composition consisting of 5 g each of compositions A, and C according to Example B16.

The colouring mixture is allowed to act on the hair for 30 minutes at about 22° C. 10 g of a 2% strength aqueous citric acid gel are then applied to the strand. After contact for 5 minutes, the strand is rinsed thoroughly, shampooed and then dried.

A strong, intense, striking coloration having good fastness to washing and fastness to rubbing properties is obtained.

### EXAMPLE 39

A dye emulsion with pH=9.8, containing:

| | | |
|---|---|---|
| direct Dye, **D1-D5 | 1.0 | |
| cetylstearylalcohol | 11.0 | |
| one of compounds (50) to (66) | 7.2 | g |
| 2,5-diaminotoluene sulfate | 2.4 | g |
| 4-amino-2-hydroxytoluene | 2.4 | g |
| 2,5,6-triamino-4- | 2.4 | g |
| hydroxypyrimidine sulfate | | |
| oleth-5 | 5.0 | |
| oleic acid | 2.5 | |
| stearic acid | 2.5 | |
| monoethanolamide | | |
| coco fatty acid | 2.5 | |
| monoethanolamide | | |
| sodium laurylsuphate | 1.7 | |
| 1,2-propanediol | 1.0 | |
| ammoniumchloride | 0.5 | |
| EDTA, tetrasodiumsalt | 0.2 | |
| perfume | 0.4 | |
| cornproteinhydrolysate | 0.2 | |
| silica | 0.1 | |

direct Dye, **D1-D5, which stands for a single direct dye with the following meaning:
D1 is Basic Yellow 87;
D2 is Basic Orange 31;
D3 is Basic Red 51;
D4 is the cationic dye of example 4 as described in WO 01/66646;
D5 is the cationic dye of example 6, compound of formula 106, as described WO 01/11708, WO 02/31056; is mixed with the same weight of 6% hydrogen peroxide solution and the mixture is immediately applied to a tress of brown hair. After 30 minutes the tress is rinsed, shampooed, rinsed and dried. The color result is a very brilliant red shade.

### EXAMPLE 40

A dye emulsion with pH 9.8, containing:

| | |
|---|---|
| direct Dye, **D1-D5 see example 39 | 0.5 |
| cetylstearylalcohol | 11.0 |
| oleth-5 | 5.0 |
| oleic acid | 2.5 |
| stearic acid monoethanolamide | 2.5 |
| coco fatty acid monoethanolamide | 2.5 |
| sodium laurylsuphate | 1.7 |
| sodiumsulphite | 1.0 |
| ascorbic acid | 0.5 |
| 1,2-propanediol | 1.0 |
| ammoniumchloride | 0.5 |
| EDTA, tetrasodiumsalt | 0.2 |
| perfume | 0.4 |
| cornproteinhydrolysate | 0.2 |
| silica | 0.1 |
| toluene-2,5-diamine sulfate | 0.07 |
| resorcinol | 0.02 |
| 2-amino-6-chloro-4-nitrophenol | 0.01 |
| 4-amino-m-cresol | 0.03 |
| 2-amino-3-hydroxypyridine | 0.001 |
| one of conpound (50) to (66) | 5 |

is mixed with the same weight of 6% hydrogen peroxide solution and the mixture is immediately applied to a tress of brown hair. After 30 minutes the tress is rinsed, shampooed, rinsed and dried.

The color result is a very brilliant ruby shade.

### EXAMPLE 43

A strand of bleached human hair is treated with 10 g of a dye composition having a pH of 9.8, comprising 5 g of a 6% hydrogen peroxide solution and 5 g of a composition A given below

Composition A

| | |
|---|---|
| oleic acid | 10.0 |
| one of compound (50) to (66) | 5.00 |
| toluene-2,5-diamine sulfate | 0.07 |
| resorcinol | 0.02 |
| 2-amino-6-chloro-4-nitrophenol | 0.01 |
| 4-amino-m-cresol | 0.03 |
| 2-amino-3-hydroxypyridine | 0.001 |
| sodium sulfite | 1.0 |
| ascorbinic acid | 0.5 |
| water | Ad 100 |

After 15 minutes, 10 g of a 12.5% citric acid gel, comprising the dye of formula (26) of example A6 according to the present application is applied on the hair and combed, so that the hair has a pH of 7. After 15 minutes the hair is washed with water, rinsed and dried. The strand has been dyed in an intensive red shade, which has a good wash and light fastness.

### EXAMPLE 44

A strand of middle blonde human hair is dyed with 10 g of a composition having a pH of 9.8, which is obtained by mixing 10 g of 6% hydrogen peroxide solution and 5 g of a composition A as given above in example 43, and 5 g of composition H50. After 15 minutes, the pH of the hair is adjusted to pH 5 by addition of citric acid. Then, 5 g of a 12.5% citric acid gel, comprising the dye of formula (26) of example A6 according to the present application, is applied on the hair and combed, so that the hair has a pH of 7. After 15 minutes the hair is washed with water, rinsed and dried. The strand has been dyed in an intensive red shade, which has a good wash and light fastness.

### EXAMPLE 45

A strand of bleached human hair is dyed with 10 g of a composition having a pH of 9.8, which is obtained by mixing 5 g of 6% hydrogen peroxide solution and 5 g of composition H50 and 5 g of a composition A given in example 43. After 15 minutes, the pH of the hair is adjusted to pH 5 by addition of citric acid. Then, 5 g of a 12.5% citric acid gel, comprising the dye of formula (26) of example A6 according to the present application, is applied on the hair and combed, so that the hair has a pH of 7. After 15 minutes the hair is washed with water, rinsed and dried. The strand has been dyed in an intensive red shade, which has a good wash and light fastness.

1. A method of coloring porous material, which comprises contacting the material being colored, with
a) a capped diazonium compound of formula 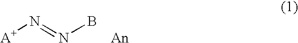
wherein
A⁺ is a cationic radical of an organic compound,
B is a radical of an unsubstituted or substituted, aliphatic or aromatic amine,
An is an anion,
and
b) optionally a coupling component.

2. A method according to claim 1, which comprises contacting the material being colored, with
a) a capped diazonium compound of formula (1)
wherein
A⁺ is a cationic radical of unsubstituted phenyl; naphthyl; thiophenyl; 1,3-thiazolyl; 1,2-thiazolyl; 1,3-benzothiazolyl; 2,3-benzothiazolyl; imidazolyl; 1,3,4-thiadiazolyl; 1,3,5-thiadiazolyl; 1,3,4-triazolyl; pyrazolyl; benzimidazolyl; benzopyrazolyl; pyridinyl; quinolinyl; pyrimidinyl; isoxazolyl; aminodiphenyl; aminodiphenylether and azobenzenyl or
A⁺ is cationic radical of a phenyl, naphthyl, thiophenyl, 1,3-thiazolyl, 1,2-thiazolyl, 1,3-benzothiazolyl, 2,3-benzothiazolyl, imidazolyl, 1,3,4-thiadiazolyl, 1,3,5-thiadiazolyl, 1,3,4-triazolyl, pyrazolyl, benzimidazolyl, benzopyrazolyl, pyridinyl, quinolinyl, pyrimidinyl and isoxazolyl, aminodiphenyl, aminodiphenylether and azobenzenyl, each of which is mono- or poly-substituted by C₁-C₄alkyl, C₁-C₄alkoxy, C₁-C₄alkylthio, quaternised ammonium radicals, halogen, e.g. fluorine, bromine or chlorine, nitro, trifluoromethyl, CN, SCN, C₁-C₄alkylsulfonyl, phenylsulfonyl, benzylsulfonyl, di-C₁-C₄alkylaminosulfonyl, C₁-C₄alkyl-carbonylamino, C₁-C₄alkoxysulfonyl or by di-(hydroxy-C₁-C₄alkyl)-aminosulfonyl, or
A⁺ is a cationic radical of an organic dye, and
B is a radical of formula —NR₆₅R₆₆, wherein R₆₅ is hydogen; or unsubstituted linear or branched C₁-C₆alkyl or linear or branched C₁-C₆alkyl, which is substituted by one or more identical or different substituent selected from the group consisting of OC₁-C₄alkyl, COOH, COO⁻, COOC₁-C₂alkyl, SO₃H, SO₃⁻, NH₂, CN, halogen and OH, O⁻; and R₆₆ is unsubstituted linear or branched C₁-C₆alkyl or linear or branched C₁-C₆alkyl, which is substituted by one or more identical or different substituent selected from the group consisting of OC₁-C₄alkyl, COOH, COO⁻,COOC₁-C₂alkyl, SO₃H, SO₃⁻, NH₂, CN, halogen and OH, O⁻; or
B is a radical of unsubstituted aniline; or a radical of unsubstituted aminonaphthalene; the radical of aniline or aminonaphthalene, wherein the phenyl or the naphthyl ring is substituted by one or more identical or different substituent selected from the group consisting of COOH, COO⁻, SO₃H, SO₃⁻, CN, halogen, SO₂C₁-C₂alkyl, unsubstituted linear or branched C₁-C₄alkyl, linear or branched C₁-C₄alkyl, substituted by OH, O⁻, COOH, COO⁻, COC₁-C₂alkyl or
SO₂-N(C₁-C₄alkyl)-(CH₂)₁₋₄SO₃H and wherein the amino radical is substituted by hydrogen, unsubstituted linear or branched C₁-C₄alkyl or linear or branched C₁-C₄alkyl, substituted by OH, O⁻, or COOH, COO⁻;
An is an anion,
and
b) a coupling component.

3. A method according to claim 2, wherein A⁺ is a cationic radical of unsubstituted phenyl; naphthyl; thiophenyl; 1,3-thiazolyl; 1,2-thiazolyl; 1,3-benzothiazolyl;
2,3-benzothiazolyl; imidazolyl; 1,3,4-thiadiazolyl; 1,3,5-thiadiazolyl; 1,3,4-triazolyl; pyrazolyl; benzimidazolyl; benzopyrazolyl; pyridinyl; quinolinyl; pyrimidinyl; isoxazolyl; aminodiphenyl; aminodiphenylether and azobenzenyl or
A⁺ is cationic radical of a phenyl, naphthyl, thiophenyl, 1,3-thiazolyl, 1,2-thiazolyl, 1,3-benzothiazolyl, 2,3-benzothiazolyl, imidazolyl, 1,3,4-thiadiazolyl, 1,3,5-thiadiazolyl, 1,3,4-triazolyl, pyrazolyl, benzimidazolyl, benzopyrazolyl, pyridinyl, quinolinyl, pyrimidinyl and isoxazolyl, aminodiphenyl, aminodiphenylether and azobenzenyl, each of which is mono- or poly-substituted by C₁-C₄alkyl, C₁-C₄alkoxy, C₁-C₄alkylthio, halogen, e.g. fluorine, bromine or chlorine, nitro, trifluoromethyl, CN, SCN, C₁-C₄alkylsulfonyl, phenylsulfonyl, benzylsulfonyl, di-C₁-C₄alkylaminosulfonyl, C₁-C₄alkyl-carbonylamino, C₁-C₄alkoxysulfonyl or by di-(hydroxy-C₁-C₄alkyl)-aminosulfonyl, or
A⁺ is a cationic radical residue of an organic dye selected from anthraquinon dye, acridine dye, azo dye, azomethin dye, hydrazomethin, benzodifuranone dye, coumarin dye, diketopyrrolopyrrol dye, dioxaxine dye, diphenylmethane dye, formazan dye, indigoid dye, indophenol, naphtalimide dye, naphthoquinone dye, nitroaryl dye, merocyanine dye, methin dye, oxazine dye, perinone dye, perylene dye, pyrenequinone dye, phthalocyanine dye, phenazine dye, quinonimine dye, quinacridone dye, quinophtalone dye, styryl dye, triphenylmethan dye, xanthene dye, thiazine dye and thioxanthene dye, and
B is a radical of formula —NR₆₅R₆₆, wherein R₆₅ is hydrogen; or unsubstituted linear or branched C₁-C₆alkyl or linear or branched C₁-C₆alkyl, which is substituted by one or more identical or different substituent selected from the group consisting of OC₁-C₄alkyl, COOH, COO⁻, COOC₁-C₂alkyl, SO₃H, SO₃⁻, NH₂, CN, halogen and OH, O³¹ and R₆₆ is unsubstituted linear or branched C₁-C₆alkyl or linear or branched C₁-C₆alkyl, which is substituted by one or more identical or different substituent selected from the group consisting of OC₁-C₄alkyl, COOH, COO⁻, COOC₁-C₂alkyl, SO₃H, SO₃⁻, NH₂, CN, halogen, OH and O⁻.

4. A method according to claim 3, wherein A⁺ is a cationic radical of an organic dye selected from azo dye, azomethin dye, hydrazomethin dye, merocyanine dye, methin dye and styryl dye.

5. A method according to claim 1, wherein there is used as a coupling component an unsubstituted or substituted acylacetarylamide, phenol, naphthol, pyridine, quinolone, pyrazole, indole, diphenylamine, aniline, aminopyridine, pyrimidone, naphthyl-amine, aminothiazole, thiophene or hydroxypyridine.

6. A method according to claim 5, wherein a coupling component is used, which is mono- or poly-substituted by amino, alkylamino, dialkylamino, halogen, alkyl, alkoxy, phenyl, naphthyl or aryloxy.

7. A method according to claim 2, wherein A⁺ is a cationic radical of a dye of formulae (7) and (8) 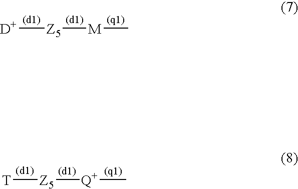
wherein
Z₅ is a biradical selected from:
—N═N—, —CR₆=N—, —N═R₇—, —NR₈—N=CR₉—, —R₁₀═N—NR₁₁—, —, CR₆═CR₆—,
wherein
R₆, R₇, R₈, R₉, R₁₀ and R₁₁, are each independently of the other hydrogen, or unsubstituted or substituted C₁-C₁₄alkyl, allyl, —C₅-C₁₀aryl, —C₁-C₁₀alkylen(C₅-C₁₀aryl),
—C₅-C₁₀arylen-(C₁-C₁₀alkyl), and
D⁺ is a radical of a cationic, aromatic, substituted or unsubstituted heterocyclic compound,
M is a biradical of an aromatic substituted or unsubstituted compound,
T is a radical of an aromatic substituted or unsubstituted compound, and
Q⁺ is a biradical of an aromatic, substituted or unsubstituted heterocyclic compound.

8. A method according to claim 7, wherein D⁺ is a radical of a cationic aromatic substituted or unsubstituted heterocyclic compound of formulae (9), (10), (10′), (10″), (11), (12) or 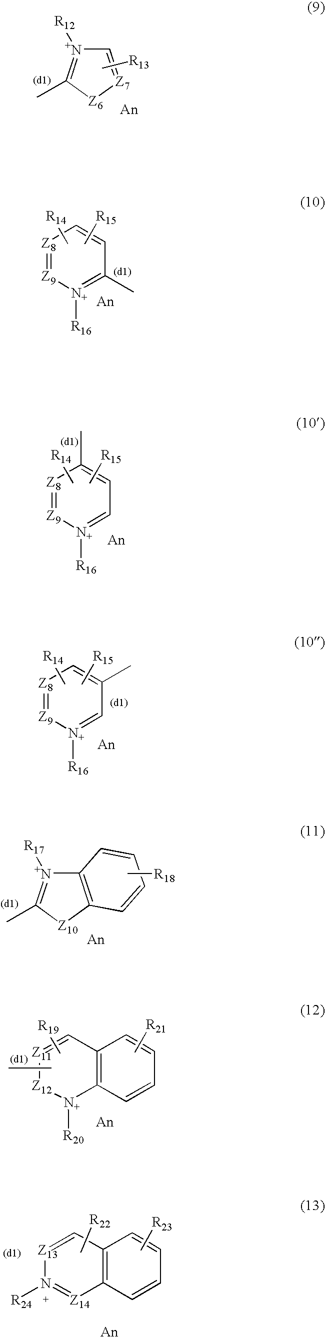
wherein
(d1) is a bond of formula (7); and
Q⁺ is a biradical of a cationic aromatic substituted or unsubstituted heterocyclic compound of formulae (14), (14′), (15), (15′), (15″), (16), (17) or (18) 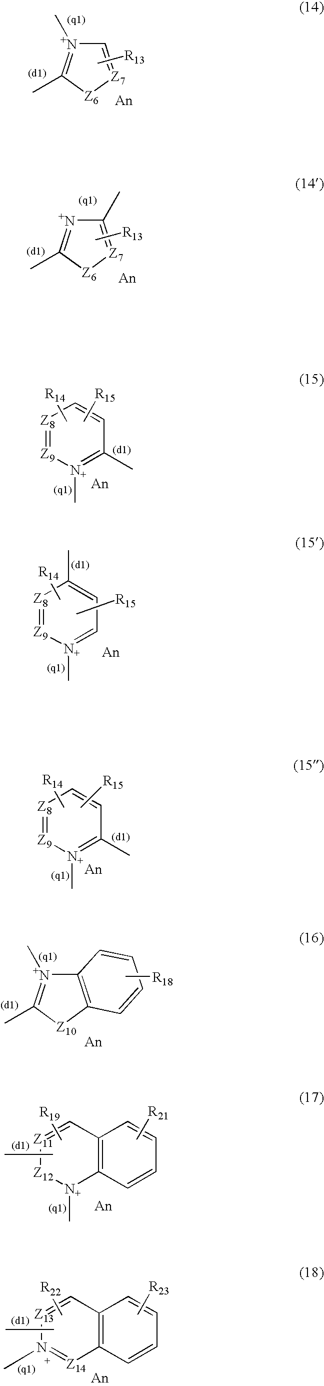
wherein
(d1) and (q1) are a bond to Z₅ of formula (8),
and
M is a biradical of formulae (19) or (20), 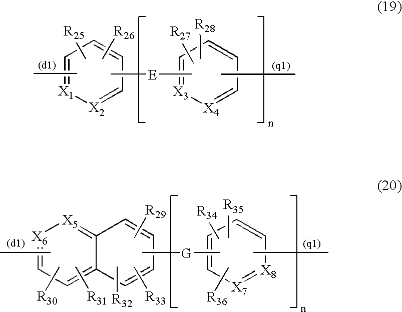
wherein
(d1) and (q1) are a bond of formula (7), and
T is a radical of compounds of formulae (21) or (22), 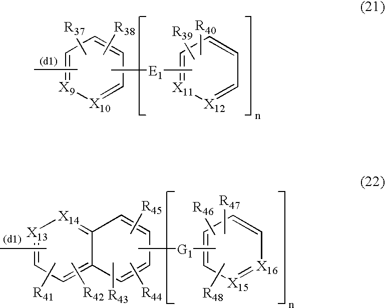
wherein
(d1) is a bond of formula (8), and
wherein
X_{1,} X_{2,} X_{3,} X_{4,} X_{5,} X_{6,} X_{7,} X_{8,} X_{9,} X_{10,} X_{11,} X_{12,} X_{13,} X_{14,} X₁₅ and X₁₆ are independently from each other N or a radical of CR₄₉,
Z₆ is O or S or a radical of NR₅₀,
Z₇, Z₈, Z₉, Z₁₀, Z₁₁, Z₁₂, Z₁₃ and Z₁₄ are independently from each other N or a radical CR₅₁;
E, E₁, G and G₁ are independently from each other —O—, —S—, —(SO₂)—, —C₁-C₁₀alkylen or —(NR₅₂)—;
R_{13,} R_{14,} R_{15,} R_{18,} R₁₉, R₂₁, R₂₂, R₂₃, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉ and R₅, are independently from each other hydrogen, halogen, C₁-C₁₄alkyl, which is saturated or unsaturated, linear or branched, substituted or unsubstituted, or interrupted or uninterrupted with heteroatoms; a radical of phenyl, which substituted or unsubstituted; a radical of carboxylic acid; a radical of hydroxy, nitril, C₁-C₁₆alkoxy, (poly)-hydroxy-C₂-C₄-alkoxy, carboxylic acid, sulfonic acid; halogen, sulfonylamino, SR₆₀, NHR₅₃ or NR₅₄R₅₅, OR₆₁, SO₂, COOR₆₂, NR₅₆COR₅₈, CONR₅₇; and
R_{12,} R_{16,} R_{17,} R_{20,} R₂₄, R₅₀, R₅₂, R₅₃, R₅₄, R₅₅, R₅₆, R₅₇, R₅₈, R₆₀, R₆₁ and R₆₂ are each independently of the other hydrogen, unsubstituted or substituted C₁-C₁₄alkyl, allyl, —C₅-C₁₀arylen-(C₁-C₁₀alkyl), —C₁-C₁₀alkylen(C₅-C₁₀aryl), C₅-C₁₀aryl, and
An is an anion.

9. A method according to claim 7, wherein D⁺ is a radical of a cationic aromatic substituted or unsubstituted heterocyclic compound of formulae (23), (24), (24a), (25), (26), (26a) or (27) 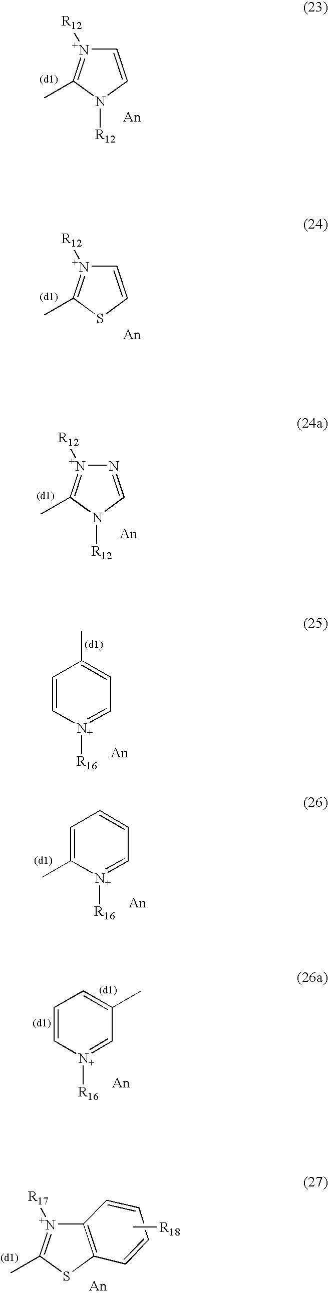
wherein
(d1) and (q1) are a bond of formula (7), and
R₁₈ is independently from each other hydrogen, halogen, C₁-C₁₄alkyl, which is saturated or unsaturated, linear or branched, substituted or unsubstituted, or interrupted or uninterrupted with heteroatoms; a radical of phenyl, which substituted or unsubstituted; a radical of carboxylic acid; a radical of hydroxy, nitril, C₁-C₁₆alkoxy, (poly)-hydroxy-C₂-C₄-alkoxy, carboxylic acid, sulfonic acid; halogen, sulfonylamino, SR₆₀, NHR₅₃ or NR₅₄R₅₅, OR₆₁, SO₂, COOR₆₂, NR₅₆COR₅₈, CONR₅₇; and
R_{12,} R_{16,} R_{17,} R₅₃, R₅₄, R₅₅, R₅₆, R₅₇, R₅₈, R₆₀, R₆₁ and R₆₂ are each independently of the other hydrogen, unsubstituted or substituted C₁-C₁₄alkyl, allyl,
—C₅-C₁₀arylen-(C₁-C₁₀alkyl), —C₁-C₁₀alkylen(C₅-C₁₀aryl), C₅-C₁₀aryl, and
An is an anion,
and
Q⁺ is a biradical of a cationic aromatic substituted or unsubstituted heterocyclic compound of formulae (28), (28a), (29), (29a), (30), (31), (31 a) or (32) 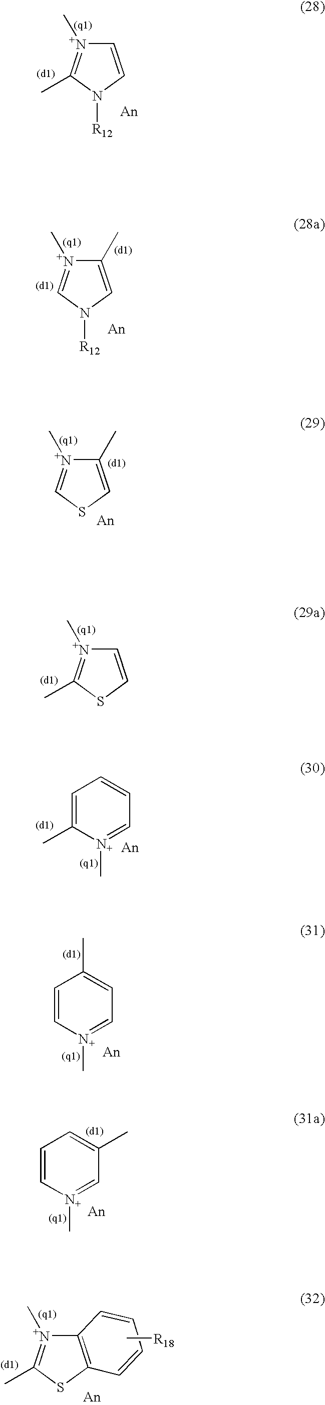
wherein
(d1) and (q1) are bond of formula (8), and
M is a biradical of formulae (33), (33a) or (33b), 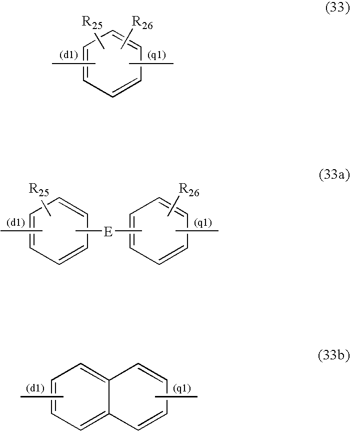
wherein
(d1) and (q1) are bond of formula (7), and
E is —O—, —S—, —(SO₂)—, —C₁-C₁₀alkylen or —(NR₅₂)—;
R₂₅, R₂₆, R₃₇ and R₃₈ are independently from each other hydrogen, halogen, C₁-C₁₄alkyl, which is saturated or unsaturated, linear or branched, substituted or unsubstituted, or interrupted or uninterrupted with heteroatoms; a radical of phenyl, which substituted or unsubstituted; a radical of carboxylic acid; a radical of hydroxy, nitril, C₁-C₁₆alkoxy, (poly)-hydroxy-C₂-C₄-alkoxy, carboxylic acid, sulfonic acid; halogen, sulfonylamino, SR₆₀, NHR₅₃ or NR₅₄R₅₅, OR₆₁, SO₂, COOR₆₂, NR₅₆COR₅₈, CONR₅₇; and
R₅₂, R₅₃, R₅₄, R₅₅, R₅₆, R₅₇, R₅₈, R₆₀, R₆₁ and R₆₂ are each independently of the other hydrogen, unsubstituted or substituted C₁-C₁₄alkyl, allyl,
—C₅-C₁₀arylen-(C₁-C₁₀alkyl), —C₁-C₁₀alkylen(C₅-C₁₀aryl), C₅-C₁₀aryl,
and
T is a radical of formulae (34) or (34a), 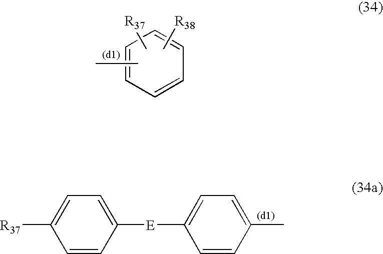
wherein
(d1) is a bond of compound of formula (8).

10. A method according to claim 1, which comprises contacting the material being colored, with
a) at least a single capped diazonium compound selected from the group of compounds of the following formulae 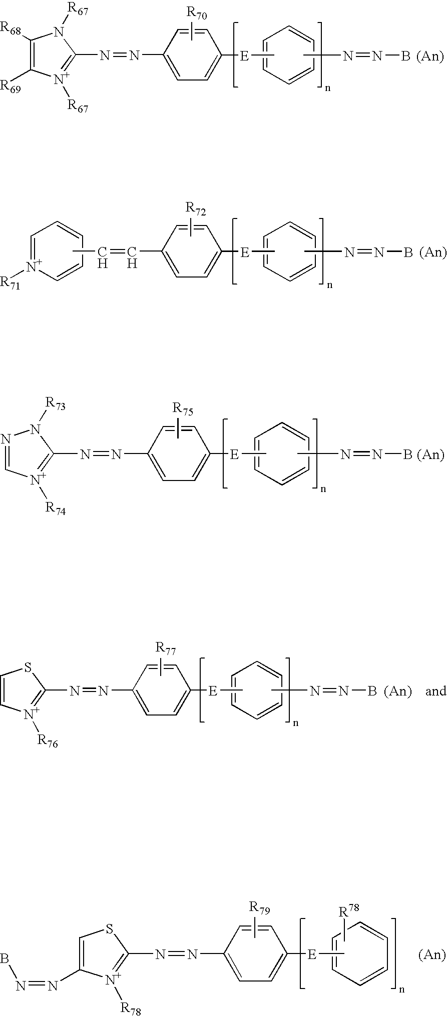
wherein
E is —O—, —S—, —(SO₂)—, CR₈₀ or a radical of —(NR₈₁)—;
R₇₀, R₇₂, R₇₅, R₇₇, R₇₈, R₇₉, R₈₀ and R₈₁ are independently from each other hydrogen,
C₁-C₁₆alkyl, which is saturated or unsaturated, linear or branched, substituted or unsubstituted, or interrupted or uninterrupted with heteroatoms, such as, by hydroxy, nitril, amino, C₁-C₂alkoxy, (poly)-hydroxy-C₂-C₄-alkoxy, di-C₁-C₂ alkylamino, carboxylic acid, sulfonic acid; a radical of phenyl, which substituted or unsubstituted; a radical of carboxylic acid; a radical of sulfonylamino, S, NH or N(C₁-C₄alkyl), O, halogen, SO₂, COO, OCO, NHCO, CONH, CON(C₁-C₄alkyl) or N(C₁-C₄alkyl)CO; or are independently from each other an aliphatic or aromatic, substituted;
R₆₈ with R₆₉ have the same meaning as R₇₀, R₇₂, R₇₅, R₇₇, R₇₈, R₇₉, R₈₀ and R₈₁ as given above, or R₆₈ with R₆₉ can build up an aromatic carbon cycle;
R₆₇, R₇₁, R₇₃, R₇₄, R₇₆ and R₇₈ are unsubstituted or substituted C₁-C₁₄alkyl, allyl, —C₅-C₁₀arylen-(C₁-C₁₀alkyl), —C₁-C₁₀alkylen(C₅-C₁₀aryl), C₅-C₁₀aryl;
B, An and n have the same meaning as defined above;
and
b) a coupling component.

11. Compounds of formula (1) 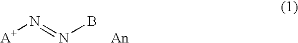
wherein
A⁺ is a cationic radical of an organic compound,
B is a radical of an unsubstituted or substituted, aliphatic or aromatic amine,
An is an anion, with the proviso that A⁺ is not a radical of formula

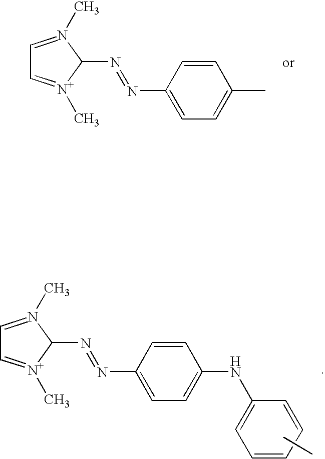

12. A composition comprising at least a single capped diazonium compound of formula (1) as defined above in claim 1 and a coupling component.

13. A composition according to claim 12 comprising in addition at least a single direct dye, at least a single oxidative dye or an oxidative agent or a combination thereof.

14. A composition according to claim 13 in form of a shampoo, conditioner, gel or emulsion.

15. A method according to claim 1 for dyeing or tinting human hair.
